(19) United States
(12) Patent Application Publication
(10) Pub. No.: US 2026/0299685 A1
(43) Pub. Date: Oct. 1, 2026

(54) Control Systems, Methods and Devices

(71) Applicant: Sensor Holdings Limited, Auckland (NZ)

(72) Inventors: Todd Gisby, Auckland (NZ); Samuel Schlatter, Auckland (NZ); Michael Andrew, Auckland (NZ); Alan Deacon, Auckland (NZ); Joren Thiessen, Auckland (NZ)

(73) Assignee: Sensor Holdings Limited, Auckland (NZ)

(21) Appl. No.: 19/489,056

(22) PCT Filed: May 30, 2024
(86) PCT No.: PCT/NZ2024/050061

§ 371 Date: Dec. 1, 2025

(30) Foreign Application Priority Data
May 30, 2023 (NZ) 800482

(51) Int. Cl. G06F 3/01; A63F 13/211; A63F 13/212; A63F 13/213; G06F 3/0346

(52) U.S. Cl. CPC G06F 3/014; G06F 3/0346; A63F 13/211; A63F 13/212; A63F 13/213; A63F 2300/8082; G06F 3/017

(57) ABSTRACT

Disclosed are wearable devices for tracking parts of the anatomy of a user, including for example the hands, fingers, feet and chest of users. In some examples of the technology the wearable device includes a processor which is configured to compare anatomical position information against a preconfigured set of anatomical positions to determine when events should be sent to a remote device. In some examples the wearable device may rely on context information to modify its behaviour or switch between, activate or deactivate operating modes of the device.

### 1. STATEMENT OF CORRESPONDING APPLICATIONS

This application claims the benefit of, and priority to New Zealand provisional patent application No. 800482 filed 30 May 2023, the entire contents of which are herein incorporated by reference.

### 2. FIELD OF INVENTION

The present technology relates to control systems, methods, and devices. The present technology may find particular application with wearable devices such as gloves, and/or in virtual/augmented/mixed reality applications, however this should not be seen as limiting on the technology.

### 3. BACKGROUND TO THE INVENTION

### 3.1. Human-Computer Interface Devices

Human-computer interface devices are devices which are peripheral to a computer and can be used to provide a limited range of inputs for controlling or triggering functions on a computer. For example, a mouse may provide two axes of movement, which generally map to the two-dimensional plane of the computer monitor, a mouse wheel to allow scrolling of the two-dimensional plane, and one or more buttons to provide limited inputs to the computer.

In many applications however it can be desirable for a greater range of control to be provided, for example when controlling objects in, or otherwise interacting in a three-dimensional space. Video game controllers exist which provide users with multiple joysticks each providing separate two axis inputs, multiple buttons, and in some cases additional analogue inputs. However, these devices are generally bulky, and still provide the user with limited interaction capabilities. For example, when interacting with a virtual object in a three-dimensional space, it may be advantageous to be able to quickly move, scale, rotate, transform, modify the geometry of the object, shade, colour or render the object, or change the positioning or perspective of the virtual camera which provides the viewport for the object. The current industry standard for selecting between these functions is typically by using a sequence of keyboard inputs, which the user typically needs to memorise, or detailed menus.

A further limitation of existing input devices is that they are not suitable for mixed-reality applications which blend the real world with a virtual environment. For example, when interacting with objects in the real world, it can be important for the user to have both hands free to pick up objects or interact with controls. It is often impractical therefore for the user to need to carry around a peripheral device such as a video game controller or keyboard while interacting with the real-world.

Controllers designed for virtual environments, such as gaming controllers, may allow for rough positional tracking, and one or more buttons to provide inputs to the virtual world. However, these provide limited control in the virtual environment, and while pointing and clicking is provided these systems still rely on menu structures which can be fiddly of cumbersome to navigate in 3D spaces. Furthermore, existing controllers used in virtual environments are generally hand-held devices, and as such the user cannot perform other functions with their hands simultaneously, which can be important, particularly in augmented reality and mixed reality applications.

### 3.2. Virtual Environments

Aspects of the present technology, relate to systems, methods, and/or devices for interacting with virtual environments. This should be understood to include:
Computer interfaces as is commonly presented on one or more two dimensional screens;
Virtual reality, such as simulated three-dimensional environments;
Augmented reality whereby computer-generated imagery is superimposed on a user's view of the real world;
Mixed reality which merges a real-world environment with a computer-generated environment such that objects may co-exist in both environments.

Extended reality it a term which covers the virtual, augmented and mixed reality applications, while throughout the present specification reference to virtual environments should be understood to include any one or more of the foregoing environments, including environments presented on a traditional two-dimensional computer screen.

### 3.3. User Tracking

One approach to addressing some of the shortcomings of traditional human interface devices, is to perform user tracking in order to identify more generally the position, orientation, and actions of a user. Various user tracking systems have been used in the past, however each of these have limitations which limit their suitability for use in certain applications.

Aspects of the present technology may seek to address any one or more of these limitations, at least partially, or at a very least provide the public with an alternative choice.

### 3.3.1. Cameras

Cameras may be used to track the position of a user's body in real time, and as such information about the position of the user's head, hands and other limbs may be available, but these systems generally require a dedicated, fixed space to work effectively. For example, one or more cameras may be positioned in the corners of a room, facing inwardly to detect occupant movements within the room. As a result, the movements of individuals within a room can be used to control or otherwise interact with objects in a virtual environment. However, these systems typically constrain the user to a fixed space which is within the viewport of the cameras, and as a result cannot be easily used in other rooms.

There are often detailed calibration steps which are undertaken as part of the setup and configuration of these camera-based systems, such as determining the relative positioning of the cameras to each other, and accounting for environmental conditions such as lighting within the room. Accordingly, it is inconvenient for these systems to be moved between rooms or environments.

User cameras may be used to address the space constraint issues at least partially. For example, in an augmented reality, or mixed reality system a user may hold a smartphone to capture a video of a scene or environment, or a head mounted camera may be used, such as is becoming more common in extended reality applications. However, since these devices need to be user-mounted they suffer from being bulky, often uncomfortable, and can have limited run time due to battery power limitations. In addition, handheld cameras, occupy the user's hands and therefore limit the user's ability to perform other functions at the same time.

A further limitation of camera-based tracking systems is that they tend to suffer from occlusion issues, whereby the cameras lose track of parts of the human body, due to being obscured from view to the user, either by the user's own body, or as soon as they interact with an object in the environment.

Traditional camera-based systems which perform finger tracking also have a number of limitations, due to the similarity of the fingers of the user's hands, it can be difficult in any given image to determine which detected finger corresponds to the which finger of the user's hand. Accordingly, these systems suffer from errors in mapping the user's fingers from the real-world to the virtual environment accurately.

It is also difficult to accurately track the movements of the user within a three-dimensional space. For example, in order to track the position of the user's hands accurately, it may be necessary to track a large number of positional variables of the user's hand, all of which are contained within a relatively small area and can be present at any location within the three-dimensional space. Accordingly, where camera tracking is used, in order to provide accurate tracking a large number of high-resolution cameras are often required, together with relatively complex software, and significant user cost, while still being vulnerable to the aforementioned occlusion issues, issues with lighting and environmental conditions and large computational requirements in order to be able to process the video images at a high frame rate.

### 3.3.1.1. Depth Cameras

Some cameras are available which include the ability to measure distance from the camera. These cameras are known as depth cameras. For example, these cameras may incorporate time-of-flight based technology to infer distance information from the camera. However, these cameras tend to have limited range capabilities, can be sensitive to reflections and transparent objects, and require relatively expensive hardware, and significant output power for their emitters, making them less suitable for portable, battery powered operation. In addition, like normal cameras depth cameras require high computational resources to process the resultant data.

### 3.3.1.2. Camera Constellations

A constellation of external cameras, normal or depth based, that track the hands directly or markers on the hands can reduce the impact of occlusion, but this comes at the expense of hyper redundancy of cameras and a large power budget, is still susceptible to the hand being occluded by the rest of the body or itself and restricts the hand tracking to a fixed location and volume.

### 3.3.2. Inertial Measurement Systems

Motion sensing electronics, such as accelerometers, gyroscopes and magnetometers may be used to track relative movements of objects with a three-dimensional space, and have relatively low weight, power consumption and do not suffer from occlusion issues in the same way that camera-based systems do.

Since these motion sensing devices do not typically have a fixed external reference, they need to perform integration of the data received to determine how the device has moved through a three-dimensional space. This approach to motion tracking is known as dead-reckoning, and is prone to drift over time, making them unsuitable for use in applications which require accuracy over time. Since these devices operate with by sampling the sensor values periodically, the accuracy of measurement can also be impacted negatively be sharp movements and impacts.

Furthermore, as these devices only track a single point in a three-dimensional space, in order to track the whole body of a user, it may be necessary to include a large number of these sensors on the user, particularly where complex tracking is required, such as in hand tracking applications.

While some devices, incorporate a number of motion sensing devices in a single package (known as Inertial Measurement Units or IMUs) these devices are still rigid, require power, and communications in order to transmit their information to an external device such as a computer, and accordingly, as the number of sensors attached to the human body increases, the more complex and in some cases uncomfortable the system becomes.

### 3.3.3. Stress and Strain Sensors

Aspects of the present technology may relate to devices comprising stress and/or strain sensors, such as capacitive sensors. Capacitive sensors may be provided in a flexible membrane such as is described in PCT publication no WO2015053638A1. These capacitive sensors are generally soft, flexible devices able to readily conform to the shape of the hand both statically and when the hand undergoes complex motion.

However, these capacitive sensors, when incorporated in a wearable device, such as a glove, they often need to be calibrated for a specific user, or a specific hand size in order to be able to accurately map the capacitance readings to hand position data. In addition, relatively complex control algorithms are required to address the non-ideal electrical properties of stretchable electrodes. Furthermore, since these types of sensors can only measure capacitance, they do not inherently provide information about the location of the user's hand in the three-dimensional environment.

### 3.3.4. Machine Learning and Artificial Intelligence

While machine learning and artificial intelligence (AI) technology may be incorporated by systems for user tracking, this is not a straightforward data processing task. The human body, and in particular the hands are relatively complex, and subject to wide ranges of variation within the population, and the environments in which they are used.

Accordingly, in order to provide user tracking technology which can work across a range of individuals, a prohibitively large dataset of information is likely to be required. This invites the subsequent issues associated with large scale data collection, in particular ensuring consistent quality and accurate and complete labelling of data. Single large models are simpler to manage and can more easily capture common or global relationships in the data, but can also lead to a lack of specialisation, are vulnerable to overfitting, and can be difficult to interpret or understand due to the high dimensionality and complexity of a single large model. Using a large dataset to train multiple smaller models can increase diversity, can handle different scenarios better by not looking for a one size fits all relationship for a range of scenarios, and there are a range of techniques for combining the predictions of multiple models that can improve overall accuracy and robustness. However, this requires the management of multiple models that can make retraining more time consuming, predictions can require additional computational overhead if multiple models must be evaluated in parallel, and the final output is dependent on the quality and diversity of the models available.

Furthermore, machine learning algorithms trained to track hands from video suffer from the extremely large interpersonal variability in hands across dimensions such as size, shape, skin colour, and whether the hand is gloved or not, contrast between the hand and the background, and whether there are multiple hands in addition to the hand of interest in the field of view. It requires prohibitively large datasets to train a machine learning model for all range of these conditions, which also severely negatively affects the ability to update the model based on new information.

The issues associated with the aforementioned technologies and techniques are particularly problematic in the context of wearable devices where power budgets are very limited, and portability is paramount.

In some conditions it is possible for each of the aforementioned techniques to recognise that hand tracking has degraded or has failed. A key metric in machine learning that is a leading indicator of this loss of performance is “confidence”. Confidence is a measure of how sure an algorithm is about its prediction. It is typically expressed as a percentage, with a higher percentage indicating a higher level of confidence. For example, an algorithm with 95% confidence is 95% sure that its prediction is correct. Confidence is calculated using a variety of factors, including the size and quality of the training data, the complexity of the algorithm, and the noise in the data. A larger and more diverse training dataset will typically lead to a more confident algorithm. A more complex algorithm will typically be less confident than a simpler algorithm. And noise in the data can make it more difficult for an algorithm to make accurate predictions, which can lead to lower confidence. However, an algorithm can have high confidence and still be inaccurate. This could be because the model is trained on a dataset that is not representative of the real world. For example, if a model is trained on a dataset of images of cats, but is then asked to classify images of dogs, the model may be confident in its predictions, but those predictions may be inaccurate. The model could also be overfit. Overfitting occurs when a model learns the training data too well and starts to memorize the data instead of learning the underlying patterns. This can lead to the model making accurate predictions on the training data, but inaccurate predictions on new data. Finally, a model can be confident but also inaccurate if the model is not given enough data to learn from. If a model is not given enough data, it may not be able to learn the underlying patterns in the data and may make inaccurate predictions.

### 3.4. Hand Tracking

Aspects of the present technology may be directed towards systems methods and devices for tracking the movement of a user's hand in a three-dimensional space. User hand tracking has each of the foregoing issues outlined in respect of body tracking, with the added complications of increased complexity, greater freedom of movement, a relatively small space on the human body for mounting tracking hardware, and a need to provide the user with the ability to use their hands during the tracking process.

The human hand is a complex object to model or track in an accurate manner. Each of the index, middle, ring, and little fingers have four joints: the Carpometacarpal (CMC), the Metacarpophalangeal (MCP), the Proximal Interphalangeal (PIP), and Distal Interphalangeal (DIP) joints. Each joint has three theoretical degrees of freedom (DOF) which can be thought of as rotation about three orthogonal axes, X, Y, and Z, however for practical purposes this can be reduced to one DOF for the DIP and PIP joints (bending), two DOF for the MCP joint (bending and splay), and one DOF for the CMC (bending). The thumb has five DOF, three DOF in the CMC, one DOF in the MCP and one DOF in the DIP, and the wrist adds a further two DOF (not including rotation about the axis of the forearm, which is controlled by the forearm). If we only consider two points on the range of motion of each degree of freedom, the minimum and the maximum, there are 2\hat{ }27 possible combinations or 134,217,728 possible hand orientations.

While not all DOF are entirely independent, when considering that each of these DOF can move continuously through maximum angular rotations of between 30 and 100 degrees depending on the DOF, and still further considering that additional DOF are required to describe the position of the hand in space relative to an external point of reference (for example, the point of view of the person the hand belongs to or that of an observer), the number of possible hand poses becomes astronomically large. This is further compounded again when the interpersonal variation in hands is taken into account: there is significant variation across the world's population along many dimensions such as size, shape, finger lengths, bone lengths, range of motion, and skin colour. Every hand is unique in some aspect or combination of aspects.

### 3.5. Object of the Invention

It is an object of the technology to provide control systems, methods and/or devices configured to address any one or more of the foregoing issues.

Alternatively, it is an object of the present technology to provide a wearable device, which can be used for user tracking purposes.

Alternatively, it is an object of the technology to at least provide the public with an alternative choice.

### 4. SUMMARY OF THE INVENTION

According to one aspect of the technology there is provided control systems, methods, and devices.

According to another aspect of the technology, there is provided wearable devices.

According to another aspect of the technology, there is provided a wearable device provided with a plurality of sensors configured to detect a position and/or orientation of the anatomy of a user, such as one or more limbs of a user.

According to another aspect of the technology, there is provided a wearable device comprising:
a body configured to receive a user's hand in use;
one or more finger compartments, configured to receive the fingers of the user's hand in use,
a plurality of sensors configured to detect information indicative of the position of the users hand, and or fingers.

In examples, the body may comprise a user contacting layer, and a non-user contacting layer.

In examples, the sensors may be positioned between the user contacting layer and the non-user contacting layer.

In examples, the finger compartments may be open-ended, while in other examples the finger compartments may be closed-ended. For example to provide fingerless, or full-finger gloves accordingly.

In examples, the wearable device may further comprise one or more active or passive markers, configured to be detected by a camera in use.

In examples, the plurality of sensors may comprise stress and/or strain sensors. For example the stress and/or strain sensors may be capacitive sensors.

In examples, the plurality of sensors may comprise one or more inertial measurement units. For example the inertial measurement units may comprise one or more of an accelerometer, gyroscope and/or magnetometer.

In examples, for one or more of the fingers in the user's hand a first sensor may be provided adjacent to a metacarpophalangeal joint, so as to detect in-use movement of the metacarpophalangeal joint.

In examples, for one or more of the fingers in the user's hand a first sensor may be provided adjacent to an interphalangeal joint, so as to detect in-use movement of the interphalangeal joint.

In examples, the wearable device may comprise a processor configured to convert a sensor value to a finger or hand position value.

In examples, the wearable device may comprise a processor configured to detect a gesture or pose, and trigger an action in response.

In examples, the wearable device may comprise a position tracking module configured to aid in tracking the position of the wearable device in a three dimensional space.

In examples, the wearable device may comprise a communications module configured to communicate hand position information, sensor readings or pose/gesture information to another device. For example the communications module may enable wireless communications such as WiFi or Bluetooth.

According to another aspect of the technology, there is provided a system for monitoring movements of a user's hand in an environment, the system comprising:
a glove configured to be worn on the user's hand, the glove comprising:
   one or more sensors configured to detect information indicative of the position of the users hand, and or fingers; and
   a communications module;
   a processor,
wherein in use the glove is configured to transmit the sensor information to the processor, and
wherein the processor is configured to determine whether the sensor information is indicative of a predetermined pose or gesture, and on detection, trigger an action.

In examples, the glove may be substantially as described herein in relation to one or more of the other aspects of the technology.

In examples, the processor may be configured to determine whether the predetermined pose or gesture is performed under one or more predetermined conditions before triggering the action. For example the one or more predetermined conditions may comprise any one or more of: a duration for the pose or gesture, a sequence of poses or gestures, context sensitivity, and/or one or more external inputs.

According to another aspect of the technology, there is provided a method of detecting poses or gestures performed in a glove, the glove comprising a plurality of sensors, the method comprising the steps of:
A) receiving sensor data from the plurality of sensors;
B) processing the data to determine a position or orientation of the user's hand;
C) determining whether the position or orientation corresponds to a predetermined pose or gesture.

In examples, the method may further comprise the step of D) triggering an action if the position or orientation corresponds to a predetermined pose or gesture.

According to another aspect of the technology, there is provided a pose detection system, which comprises at least one memory having computer readable computer instructions, and at least one processor for executing the computer readable instructions. The computer readable instructions including the steps of receiving sensor data from a plurality of sensors, and either A) communicating the sensor data to an external processor, or B) processing the sensor data to determine hand position data indicative of a pose.

According to another aspect of the technology, there is provided a wearable device comprising:
a body configured to receive a part of the anatomy of a user;
one or more sensors operatively connected to the body that are configured to provide data relating to the location, orientation and/or configuration of the body in use;
a processor configured to process the data to determine anatomical position information; and
a communications module operatively connected to the processor, and configured to
communicate with at least one remote device,
wherein the processor is configured to compare the anatomical position information against a pre-configured set of anatomical positions linked to one or more events, to determine whether the anatomical position information corresponds to one of the pre-configured anatomical positions,
wherein the processor receives context information from any one or more of the remote device, the events sent to the remote device; and/or from existing context information and anatomical position data, and wherein the processor uses the context information together with the anatomical position information to determine whether the processor should send an event of the linked one or more events to the remote device via the communications module.

According to another aspect of the technology, there is provided a wearable device comprising: a body configured to receive a part of the anatomy of a user;
one or more sensors operatively connected to the body that are configured to provide data relating to the location, orientation and/or configuration of the body in use;
a processor configured to process the data to determine anatomical position information; and
a communications module operatively connected to the processor, and configured to communicate with at least one remote device,
wherein the processor is configured to operate in:
   a first mode in which the anatomical position information is provided to the communications module for communication with the remote device; and
   a second mode in which the processor compares the anatomical position information against a pre-configured set of anatomical positions, wherein when the anatomical position information is determined to correspond to a pre-configured anatomical position, the processor is configured to send an event to the communications module for communicating the event to the remote device.

In examples, the processor may be configured to use a first algorithm or model with a first piece of context information, and a second algorithm or model to process the data with a second piece of context information.

In examples, the first algorithm or model may be an AI model trained to detect a first range of anatomical positions.

In examples, the second algorithm or model may be an AI model trained to detect a second range of anatomical positions.

In examples, the wearable device may be configured to communicate with a second wearable device via the communications module.

In examples, the data provided by the one or more sensors may be capacitance readings relating to a configuration of the wearable device as a result of deformation in the body of the wearable device due to the user's anatomical positioning within the wearable device.

In examples, the anatomical position information may comprise hand position information, the hand position information comprising information on the relative positioning of each of the fingers of the user with respect to the palm of the hand of the user.

In examples, the anatomical position information may include hand position information, the hand position information comprising information of the amount of bend in any one or more of the scapho-trapezium/trapezoid, the carpometacarpal, the metacarpophalangeal, the proximal Interphalangeal, and distal interphalangeal joints.

In examples, the anatomical position information comprises location and/or orientation information provided by a sensor in the form of an inertial motion unit, accelerometer, gyroscope or magnetometer.

In examples, the processor may be configured to send the event via the communications module to the remote device.

In examples, the processor may be configured to determine whether the anatomical position information corresponds to a pre-configured anatomical position using a machine learning model.

In examples, the machine learning model may be configured to determine whether the anatomical position information corresponds to a pre-configured anatomical position using a neural network.

In examples, the neural network may provide a confidence score indicating the likely match to any one of the pre-configured anatomical positions.

In examples, the processor may be configured to determine whether the anatomical position information corresponds to one of the pre-configured set of anatomical positions when the confidence score exceeds a predetermined threshold.

In examples, the processor may be configured to send the event when the confidence score for one of the pre-configured anatomical positions is higher than the confidence score for any other of the pre-configured anatomical positions.

In examples, the processor may be further configured to switch between, or simultaneously activate a first mode in which anatomical position data is communicated with the remote device, and a second mode, wherein the events are communicated with the remote device.

In examples, the processor may be configured to switch between, enable or disable either of the first mode and the second mode when a sequence of anatomical positions are detected which correspond to a pre-configured sequence of anatomical positions associated with a change of operating mode.

In examples, the processor may be configured to send the event or update the context if the pre-configured anatomical position is detected for a predetermined period of time.

In examples, the event may comprise one or more of: a keypress; a multi-media command; an animation event; an augmented/virtual/mixed reality event; displacement on one more axes; anatomic position and/or orientation data; a biometric identification event; an authorization event; or a user-defined event.

In examples, the event may comprise orientation information from an inertial motion unit, accelerometer, gyroscope or magnetometer.

In examples, the first mode and second mode may be active simultaneously.

In examples, the first mode and second mode may be active independently of each other.

In examples, the data may be provided by the one or more sensors are capacitance readings relating to the configuration of the device as a result of an amount of deformation in the body of the wearable device due to the user's anatomical or finger positioning within the wearable device.

In examples, the processor may be configured to use one or more machine learning models to process the data to determine anatomical position information.

According to another aspect of the technology, there is provided a processor-implemented method of controlling a remote device using at least one wearable device which comprises, a body configured to receive a part of the anatomy of a user, one or more sensors operatively connected to the body that are configured to provide data relating to the location, orientation and/or configuration of the body in use, a processor configured to process the data to determine anatomical position information, and a communications module operatively connected to the processor, the method comprising the steps of:
in a first mode of operation:
   A) processing the data from the one or more sensors to provide anatomical position information;
   B) comparing the anatomical position information to a pre-configured set of anatomical positions linked to one or more events;
   C) determining when the anatomical position information corresponds to a pre-configured anatomical position;
   D) communicating the corresponding events to the remote device using the communications module to thereby control the remote device;
and in a second mode of operation:
   E) processing the data from the one or more sensors to provide anatomical position information; and
   F) communicating the anatomical position information to the remote device using the communications module to thereby control the remote device.

In examples, one or more of the pre-configured set of anatomical positions may provide the processor with context information about the intended behaviour of the user.

In examples, with a first context the processor may be configured to use a first algorithm or model to process the data and with a second context, the processor may be configured to use a second algorithm or model to process the data.

According to another aspect of the technology, there is provided a wearable device comprising:
a body configured to receive a part of the anatomy of a user;
one or more sensors operatively connected to the body that are configured to provide data relating to the location, orientation and/or configuration of the body in use;
a processor configured to process the data to determine whether anatomical position information corresponds to at least one of a set of predetermined anatomical positions each of the predetermined anatomical positions being linked to one or more events; and
a communications module operatively connected to the processor, and configured to communicate with at least one remote device,
wherein the processor is configured to receive context information from the remote device, and based on the context information enable or disable one or more of the events, such that after receiving a first piece of context information, the processor is configured to select from a first list of events corresponding to the predetermined anatomical positions, and after receiving a second piece of context information the processor is configured to select from a second list of events corresponding to the predetermined anatomical positions.

In examples the context information may include information about an application running on the remote device.

In examples, the processor may receive context information from one or more of the events.

According to another aspect of the technology, there is provided a wearable device comprising: a body configured to receive the a part of the anatomy of a user;
one or more sensors operatively connected to the body that are configured to provide data relating to the location, orientation and/or configuration of the body in use;
a processor configured to process the data to determine anatomical position information; and
a communications module operatively connected to the processor, and configured to communicate with at least one remote device,
wherein the processor is configured to operate in:
   a first mode in which the anatomical position information is provided to the communications module for communication with the remote device; and
   a second mode in which the processor compares the anatomical position information against a pre-configured set of anatomical positions linked to one or more events,
wherein the processor receives context information from any one or more of the remote device, the events sent the remote device and/or from existing context information and anatomical position data, and wherein the processor uses the context information to determine whether the processor should send an event to the remote device via the communications module

It should be appreciated that the systems methods and devices described herein may be performed in a processor which is configured to execute machine-readable instructions stored on a memory. The memory may include any form of non-volatile memory for storing the machine-readable instructions, including but not limited to flash memory, battery powered RAM, read only memory (such as electrically erasable programmable read-only memory EEPROM), as well as volatile memory such as RAM. These components are typically provided on a printed circuit board assembly (PCBA) as should be familiar to those skilled in the art.

In any one or more of the examples described herein the anatomy of the user may relate to a hand or foot of the user, and the anatomical position information may be hand or foot position information and the body may be provided as a part of a glove or sock.

Accordingly, the present technology can provide one or more advantages over existing systems including:
Seamless control of remote devices while keeping the user's anatomy free to interact with other objects;
Reduced false activation of commands by requiring specific context or operating modes to be selected first.
Enhanced precision, and/or enhanced responsiveness depending on the application, operating mode, or context of the actions being performed;
The ability to receive and adapt a control scheme based on context received from an external device; and
The ability to update context on the wearable device without needing communication from an external device.

Further aspects of the technology, which should be considered in all its novel aspects, will become apparent to those skilled in the art upon reading of the following description which provides at least one example of a practical application of the technology.

### 5. BRIEF DESCRIPTION OF THE DRAWINGS

One or more embodiments of the technology will be described below by way of example only, and without intending to be limiting, with reference to the following drawings, in which:

FIG. 1 shows a perspective view of a user tracking system configured to track the position of a user in a real-world space;

FIG. 2A shows an example of a user wearing a motion capture suit, and the corresponding representation of the user's skeleton in a virtual environment;

FIG. 2B shows an example of a point cloud providing a rough outline of a toroid;

FIG. 3A shows a first example of a system diagram according to one aspect of the technology;

FIG. 3B shows a second example of a system diagram according to another aspect of the technology;

FIG. 3C shows a further example of a system diagram according to another aspect of the technology;

FIG. 3D shows an example of a rear view of a wearable device in accordance with one aspect of the present technology;

FIG. 3E shows a side view of the wearable device of FIG. 3D;

FIG. 3F shows a system diagram for a wearable device in accordance with the present technology;

FIG. 3G shows a block diagram of an electronics housing in accordance with one example of the technology;

FIG. 4 shows an example of a capacitive sensor, and method of making the capacitive sensor, according to an aspect of the present technology; and

FIG. 5 shows a system overview of how the present technology may utilise pose containers and groups to enable more accurate pose detection, and/or reduce the computational burden of pose detection.

FIG. 6 shows examples of hand poses corresponding to the American Sign Language (ASL) alphabet.

FIG. 7 shows an example flow diagram for a method of sending commands based on wearable device data in accordance with one example of the technology.

FIG. 8 shows a schematic diagram of a processing system in accordance with one example of the technology.

### 6. BRIEF DESCRIPTION OF PREFERRED EMBODIMENTS OF THE INVENTION

### 6.1. Overview of User Tracking Systems

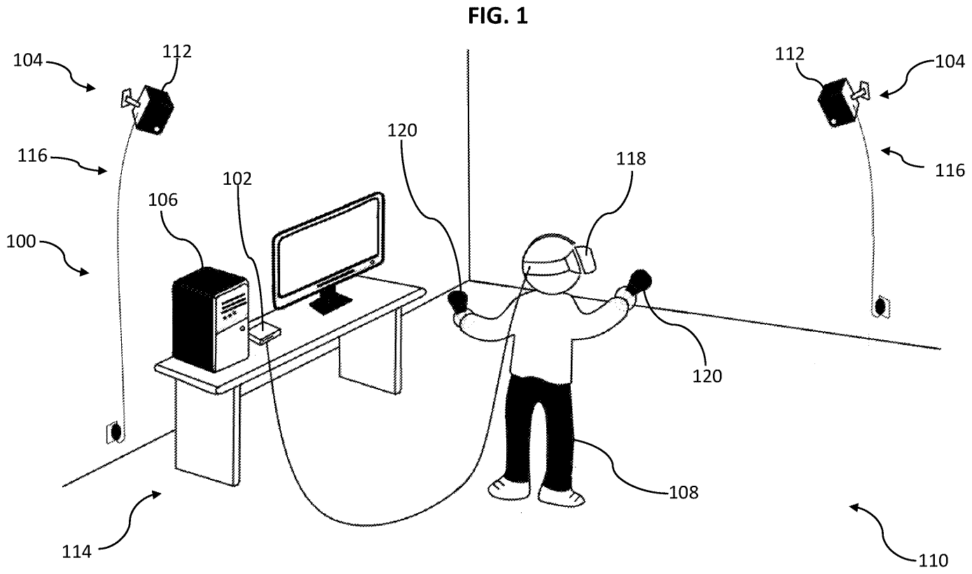

In some forms of the technology, user tracking systems 100 are provided. FIG. 1 shows a first example of the technology, wherein the user tracking system 100 comprises a user position tracking module 102, coupled to one or more sensors 104, and a processor 106 configured to receive position information from the user position tracking module 102.

In use, the sensors 104 are configured to capture information about the user's 108 position within the environment 110. For example, in the illustrated environment the sensors 104 comprise cameras 112 which are raised from a surface 114 of the environment, such as being mounted on a wall 116 or stand (not shown). By raising the cameras 112 up above a surface the user 108 will be walking on, it may be possible to reduce the likelihood that the cameras field of view will be blocked or that their view of the user 108 is occluded.

In the example of FIG. 1, the environment has been configured as an extended reality environment, such as a virtual reality (VR), augmented reality (AR), or mixed reality environment (MR). While not essential to the technology, the user 108 in the illustrated example is equipped with an extended reality headset 118 which may comprise any one or more of a display, a sound device, microphone, inertial measurement systems and/or one or more cameras.

In use as the user 108 moves through the environment, the sensors 104 can track the position of the user 108, for example where cameras 112 are used by taking a continuous sequence of photographs, or video showing the position of the user.

In some examples of the technology, the sensors 104 may be configured to track specific shapes or colours presented within the environment as a way of reducing the computational complexity of the captured images. For example, in FIG. 1 the user 108 is provided with handheld controllers 120, which the sensors may be specifically configured to track the position of. For example, the controllers 120 may have a distinct recognisable shape which is unlikely to appear in other locations within the image. By using distinct shapes, it may be possible to pre-program the user position tracking module 102 with information about the shapes, such as their geometry and colour, which allows for simplified tracking by being able to determine distance from the sensors without requiring expensive depth sensing cameras, for example by factoring in the properties of the camera, and size of the shape in the resulting image.

In another example of the technology, the controllers 120, and or may be configured to emit a light of a particular frequency which the sensors can detect, including for example light in the non-visible portion of the electromagnetic spectrum, such as infra-red or ultraviolet light. The foregoing passive and active techniques may be referred to motion capture (“mocap”) markers as should be known to those skilled in the art.

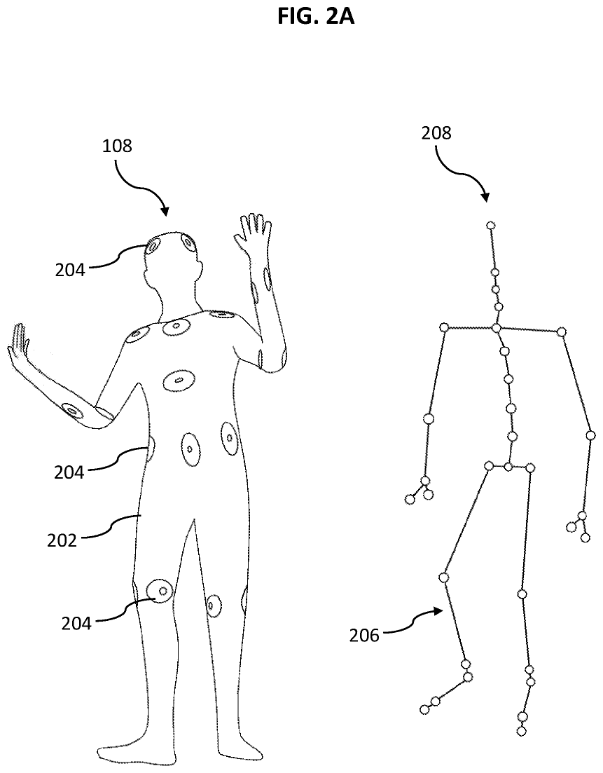

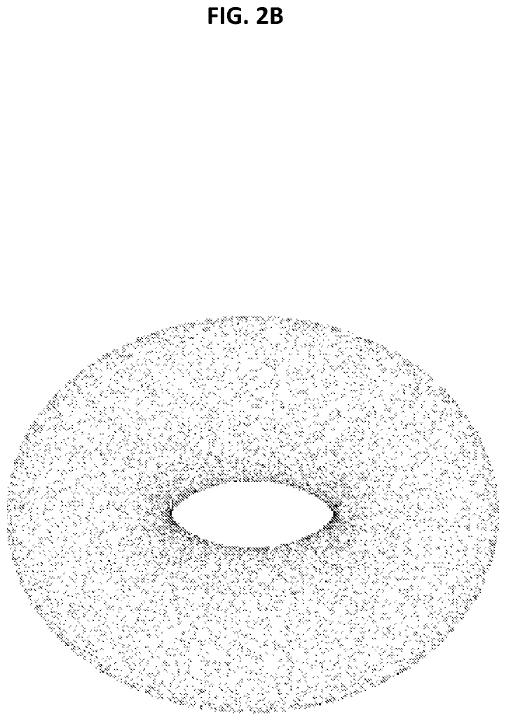

For example, as illustrated in FIG. 2A in some motion capture applications, such as those used in film and television, actors may wear a bodysuit 202 which includes a series of distinct markers 204 which the sensors 104 are configured to detect, in order to map these locations to corresponding bones 206 in a three-dimensional model. Accordingly, it can be desirable for motion capture markers to be positioned at either end of the bones within the human body, such that the corresponding bone in a “rigged” skeleton 208 can be mapped accordingly.

The user positional information may be provided by the position tracking module 102 to a processor 106. For example, the positional information may be a sequence of images which the processor 106 performs processing on to extract the required positional information, or alternatively the positional information may be provided to the processor in a format which is ready to use, such as a series of three-dimensional co-ordinates corresponding to key features of the user. For example, the three-dimensional co-ordinates may comprise the position and/or rotation of the one or more handheld controllers 120, the extended reality headset 118, or key points of the user's body corresponding to joints or bones as illustrated in FIG. 2A such as the location of the hands, feet, knees, elbows, pelvis, shoulders and arms etc.

In some examples, the positional information may comprise a point cloud, or other three-dimensional representation of the points on the user's body. For example, FIG. 2B shows a representation of a torus using a point cloud. Where point clouds are used, it may be possible to determine the overall outline of the user's body, including for example relative hand positioning. However, doing so may be computationally expensive, and as in the previous examples, these camera-based systems are all vulnerable to occlusion.

In addition, in the illustrated example, the point cloud shown in FIG. 2B are points corresponding to the user's body, to obtain this point cloud, it can be necessary to perform further signal/image processing techniques, such as removing data points corresponding to images in the foreground or background of the image based on distance, or other image processing techniques. Accordingly, point cloud processing is computationally expensive, and typically not suitable for use in high frame rate applications, or power/cost sensitive applications.

### 6.2. Applications of the Technology

Once the tracked positional data has been determined, there are a number of possible applications for the technology. In many applications, such as animation, film and gaming, the objective of the user tracking system may be to extract information which can be mapped to a skeleton in 3D software, such that movement of the user causes corresponding movement of the skeleton or rigged model surrounding the skeleton in the virtual environment. For example, the technology may be used to:
Add natural human movement to animated characters;
Put the user into a different environment, such as an environment which would be expensive or dangerous to reproduce non-digitally; and
Move a character through and or interact with virtual objects in a three-dimensional environment, such as in a VR game.

Accordingly, in some examples of the technology, it can be advantageous to extract a minimal set of information required to determine the bone positions of the user, thereby reducing the computational processing power required, and potentially reducing cost, bulk and power draw requirements of the system.

In some situations, the positional information described herein may be mapped to one or more poses or gestures. In general terms, references to poses here should be understood to mean a specific shape or orientation of a body part (such as a body part which is equipped with a wearable device as described herein). In contrast, a gesture is defined as a predetermined motion, and may include pose information. For example, in the game rock, paper scissors, each of the rock, paper and scissor hand positions could be considered poses while the action of performing the pose, i.e., extending the pointer and middle finger to create the scissor pose would be considered a gesture.

For example, when the user opens their hand palm forward towards the sensor, this pose may be detected as a “stop” command, which can be used to trigger an action within a system. For example, in a gaming console, this gesture could be used to pause the game, or when streaming video, it could be used to stop playback, pause playback or to mute the audio.

While these large obvious gestures may be detectable by some systems, they can be prone to false positives, such as the user reaching out to touch something within the virtual environment. As the ability to tracks the user's body and hands improves the user is able to perform more precise accurate movements, and as such it can be advantageous for pose detection techniques detect more subtle poses. For example, it may be desirable for a user to perform a subtle gesture, such as raising or lowering a single finger to perform a specific action, for example zooming a camera in or out, while the rest of the hand or hands may be manipulating other functions of the system.

One feature of the present technology is therefore methods, hardware and systems for triggering actions with subtle gestures and poses, and using one or more conditions to ensure that the actions are not inadvertently triggered under the wrong circumstances.

A further limitation of existing gestural detection systems is accounting for the aforementioned issues relating to occlusion, while balancing the computational costs of the positional detection systems, which increases as the amount of data being processed increases. For example, in portable battery powered systems, it can be beneficial provide the control and gesture detection while using as little processing power (and therefore energy, and battery capacity in portable systems) as possible.

Accordingly, aspects of the present technology described herein are related to providing any one or more of: systems methods and devices for providing positional information which is readily able to be converted to gestural information, systems methods and devices for sending control commands based on gestures, and systems methods and devices for providing user mappable gestural control in user tracking systems.

### 6.1. Hardware

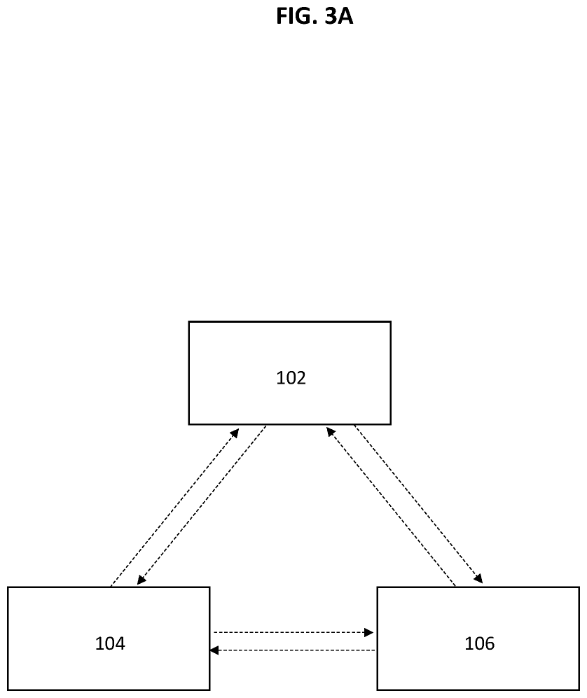

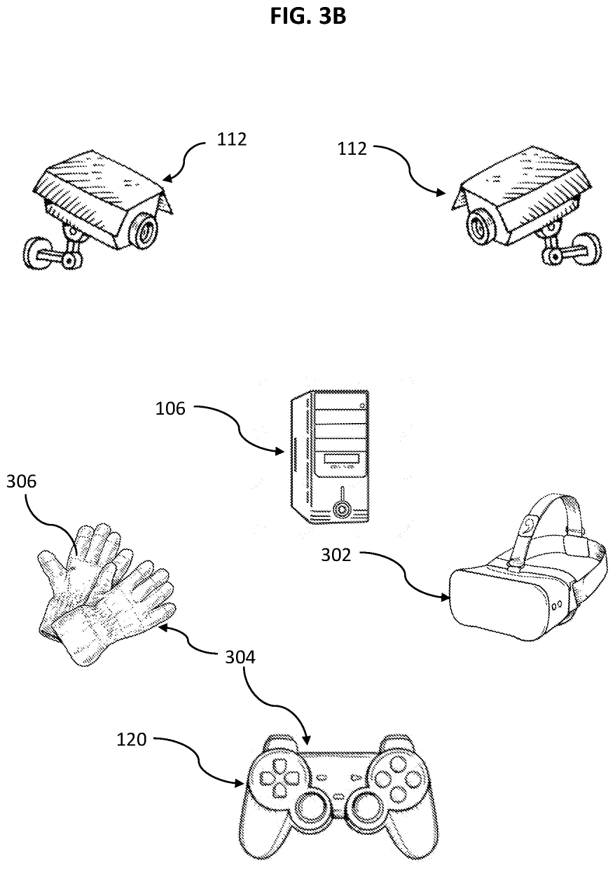

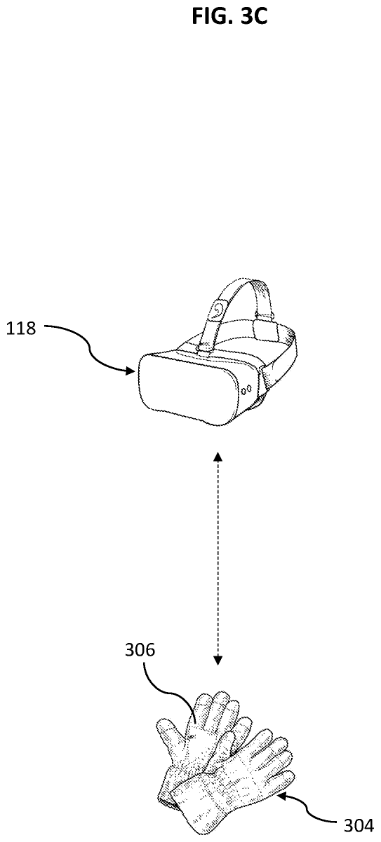

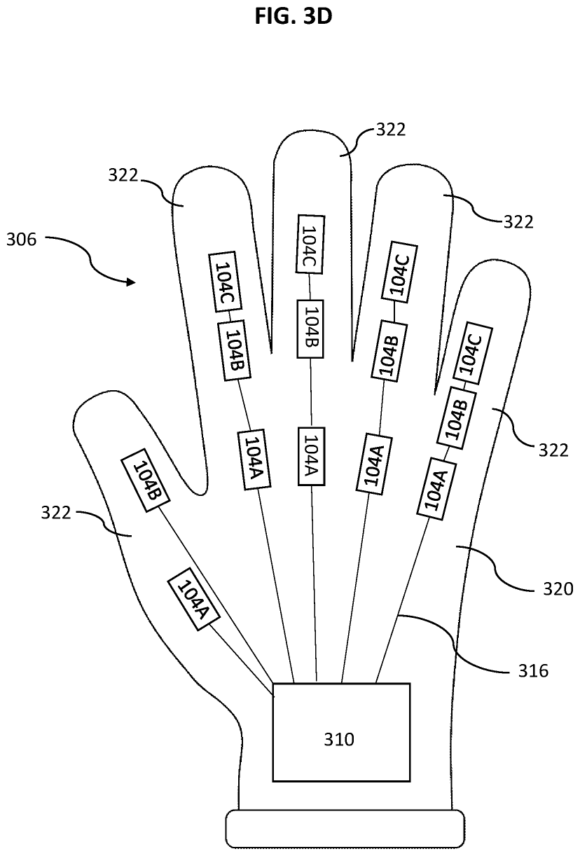

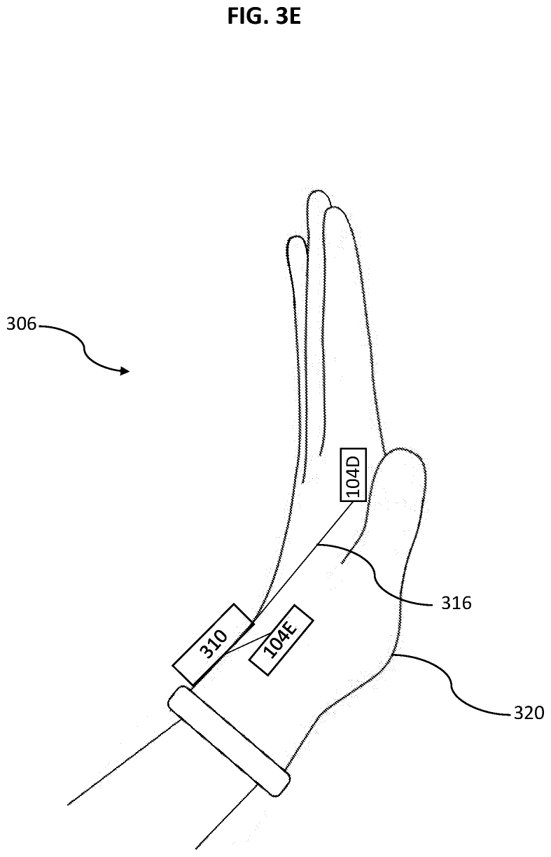

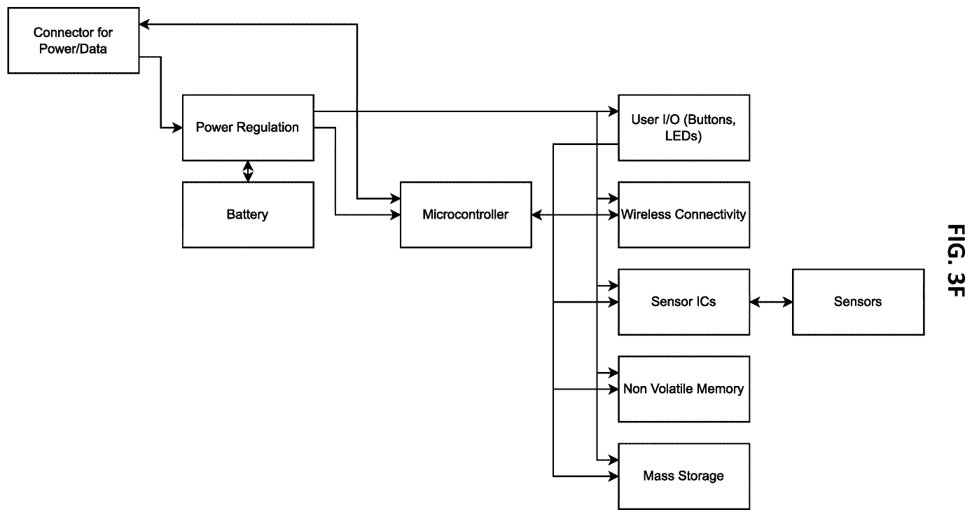

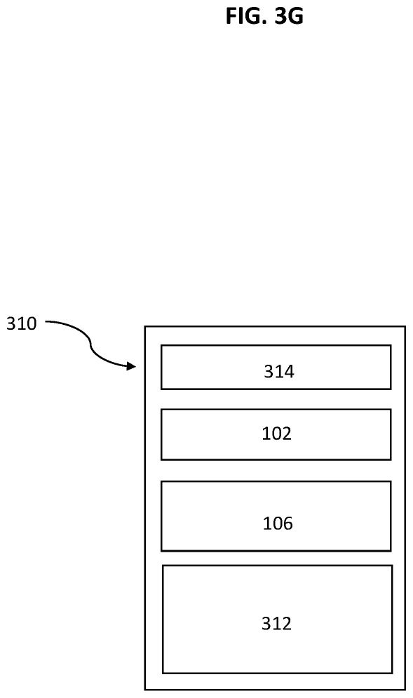

FIGS. 3A to 3D show exemplary system diagrams according to various aspects of the technology. For example, FIG. 3A shows of one example of hardware used in a user tracking system 100 as described herein. In this example, the user tracking systems 100 may comprise one or more position tracking modules 102, one or more sensors 104, and one or more processors 106.

Each of the foregoing will be described in greater detail. However, it should be appreciated that any one or more of the components described herein may be provided in a single device or module. For example, the position tracking modules 102 may comprise the sensors 104, or both the sensors 104 and processors 106.

Additionally, it should be appreciated that position tracking modules 102, one or more sensors 104 and one or more processors 106 may be configured to communicate with each other using any methods known to those skilled in the art. For example, these components may be electrically connected to one another, such as using conductive traces on a printed circuit board (PCB) or one or more wires, alternatively they may be configured to communicate with each other using one or more wireless communications, such as Bluetooth™. In a further example of the technology, any one or more of the components described herein may be configured to read and/or write to a shared memory device, such as a random-access memory (RAM), or non-volatile storage device such as an electronically erasable programmable read only memory (EEPROM), flash storage device, hard disk, or solid-state memory device.

In a first example of the technology shown in FIG. 3A, a processor 106 is configured to communicate with and receive information from one or more sensors 104 (such as stress/strain sensors) and optionally a position tracking module 102 (such as an inertial motion unit), in order to determine hand position information.

The processor 106 on receiving the sensor data, may process the data to extract hand position information, such as the positions of any one or more of the fingers of the user with respect to the hand (such as the palm or dorsum/back of the hand) of the user, and optionally the position of the hand of the user in a three-dimensional space.

In some examples, this hand position information may be sent to a remote device such as a computer, console, mobile phone or VR headset via a communications module 314 as described herein. However this should not be seen as limiting, and in some examples, the hand position information may be stored on the wearable device (such as on non-volatile memory, or battery-backed volatile memory) within the wearable device, for later download and analysis.

In some examples the processor 106 may be configured to perform an action based on the positional information, such as updating the corresponding positional information of a corresponding virtual object (such as a skeleton, or a digital twin) on a remote device. In other examples the processor 106 may be configured to determine whether specific, pre-configured hand positions are present in the hand position information, and where these are present the processor 106 may be configured to send specific commands to the remote device via the communications module. For example on detecting a pre-configured hand position which indicates that an application running on a remote device should pause, the processor may directly send a pause command to the remote device.

In some examples, the processor 106 may be configured to communicate with the sensors 104, and/or position tracking module 102 or vice versa. For example, the processor may request positional information from the position tracking module 102 as required, or the positional information may be provided periodically. In other examples, the processor 106 may be configured to communicate with the sensors directly. For example, to enable or disable sensors, or change one or more operating parameters of the sensors, such as the sampling rate.

Another example of the technology is illustrated in FIG. 3B. In this example, a processor 106 in the form of a computer may be configured to connect to any one or more of: sensors 104 such as cameras, a display 302 such as a computer monitor or VR headset 118, peripheral input devices 304 such as handheld controllers 120, and wearable devices 306.

For example, the processor 106 may be configured to render a virtual environment and present the environment to the user 108 via the display 302. When cameras 112 are included, the cameras may be configured to capture a series of images, which can be processed by a positional tracking module 102, in order to determine the position and/or rotation of the user or key features of the user, such as the hands, arms feet etc. For example, the positional tracking module 102 may be provided by the camera(s), i.e., the cameras may be configured to take photos and/or video and process the photos and/or video to extract the position and/or rotation of the user or key features of the user 108, alternatively the images and/or video may be provided to the processor 106, and the processor 106 may comprise the position tracking module 102 configured to extract the positional and/or rotational information from the images/video.

Accordingly, as the user moves, in a three dimensional environment, the processor may process data from one or more sensors to determine hand position information. In some examples a positional tracking module 102, may also track the position and/or orientation of the user in the real world, such as the position of the user within a room.

Depending on the user's movements and hand position information, the processor 106 may be configured to affect one or more actions either in the real world, or in a virtual environment. For example a predefined movement or hand position may result in turning the lights in a room on or off, pausing playback on a tv, or shooting a gun in a video game.

It should be appreciated that movement or hand position information obtained from the wearable device may be configured to affect an action in a real-world or virtual environment at any scale. For example, a character or avatar in the virtual environment may move in a corresponding manner to the person in the real world, such as 1:1 movement or any other suitable movement including faster movement i.e., greater than 1:1 such as 1:2 or slower, more precise movements such as less than 1:1 such as 1:0.1. For example the movement of the user may be configured to control a real-world device such as a surgical robot, where for example precise movements are desirable. This can allow for remote surgery to be performed for example.

In some examples, the processor 106 may not make any corresponding changes in the virtual environment until a specific movement, gesture or sequence of movements or gestures is detected. For example, when presenting a video to the user, movement of the user may have no effect unless a specific action is presented, such as a “stop” or “pause” gesture, or for example if the user were to walk out of the field of view of the cameras.

In examples of the technology which use wearable devices 306, the camera's may be configured to track the location of the wearable device 306, rather than the specific features of the user 108. For example, the wearable device 306 may comprise one or more markers (such as active or passive markers, such as reflective markers) configured to facilitate simple detection by the position tracking modules 102. Examples of passive and active markers should be known to those familiar with motion capture technologies used in film and media.

In some examples of the technology, the wearable device 306 may comprise sensors 104 configured to information to the processor 106. For example, where the wearable device 306 comprises strain and/or stress sensors such as the capacitive sensors described herein it may be beneficial for the wearable device to include a processor 106, which in use converts the sensor readings (i.e., capacitance values) to hand positional information such as finger positions relative to the palm of the hand of the user.

In some examples of the technology, the wearable device 306 may further comprise sensors 104 such as inertial movement sensors configured to communicate hand position information in a three-dimensional space. For example, accelerometers, gyroscopes and/or magnetometers may be used to track the position, and/or relative movements of the user's hand in space.

In some examples of the technology, it may be beneficial to combine positional information provided from a wearable device 306 with positional information provided from one or more cameras, such as wall mounted, or headset mounted cameras. For example, the one or more cameras, may be used to determine the position of the user's hand in an environment, while the one or more sensors mounted within the wearable device may be configured to communicate information relating to the finger positions of the user. By using a plurality of positional information sources, it may be possible to track the user's positional information more accurately within the environment.

While the example of FIG. 3B illustrates one example of the system where a central processor 106 is provided this should not be seen as limiting on the technology. For example, the system 100 may configured to operate as a mesh network, and any one or more of the components described herein may be configured to communicate with any of the other components of the system. For example, a first wearable device 306 may be configured to communicate with a second wearable device 306, so as to communicate hand position data therebetween. This approach can advantageously enable one or more of the processors 106 to affect an action based on the hand position information from both wearable devices.

In other examples, the one or more cameras 112 may be configured to communicate directly with the headset 118, in order to share information therebetween. For example, the headset may comprise inertial sensors 104 which provide information about the movements of the user's head, this information may be combined with image or video information from one or more cameras in order to get an accurate model of the position of the user's head in the environment. Accordingly, in this example, the headset 118 may comprise a positional tracking module 102 configured to provide positional information to the processor 106, wherein the headset 118 communicates the positional information from one or more sources (which could include from wearable devices) to the processor.

FIG. 3C shows a further example of the technology wherein the processor is provided within a headset 118. In this example, the headset 118 is configured to generate a virtual environment and present this to the user. The headset 118 may also communicate with one or more peripheral devices such as handheld controllers, wearable devices and cameras in order to allow the user to interact with the environment. In some examples, the headset 302 may comprise cameras 112, configured to track the position of the user's hands in the three dimensional space.

It should further be appreciated that while the full components of the system have not been illustrated in these examples, common electronic components may also be provided including for example a power source, configured to power the one or more electronic devices. For example, the power source may be a DC power source such as a battery, or an AC power source such as is provided from an electrical grid, or transformer. Furthermore, examples of the technology may comprise one or more regulators configured to regulate the voltage and or current supplied to the one or more components described herein.

For example, FIG. 3F shows an exemplary connection diagram for a wearable device in accordance with the present technology. As shown, the device may comprise a connector for power and/or data. For example, the connector may allow for charging or the internal battery between use, data transfer during use, a diagnostic connection, and/or an interface to allow for the firmware or software on the device to be updated.

The device comprises an internal battery, with an optional power regulation circuit. For example, the power regulation circuit may comprise one or more of a linear or switching regulator configured to maintain a regulated voltage output.

In the illustrated example the processor 106 is indicated as a microcontroller (i.e., a device which combines a processor with memory and one or more programmable inputs and outputs). However this should not be seen as limiting. For example the processor, and memory may be separate components, such as is common in personal computers.

In some examples of the technology the wearable device may comprise:
User inputs and outputs (I/O) such as buttons to control the device (switch the device on/off, pair Bluetooth, change operating modes etc.);
Wireless connectivity, such as a WiFi or Bluetooth connection to allow for the transfer of information in use, such as hand/finger capacitance or position values.
One or more sensors 104 as described herein, for example this may comprise capacitive sensors, and one or more sensor IC's to facilitate the measurement of capacitance from the capacitive sensors.
Non-volatile memory, for example to store the firmware or software which is configured to control the operation of the wearable device in use.
Mass storage, for example to store configuration settings, usage information, user definable conditions or any other suitable information which may be of use to the software or firmware during use.

### 6.1.1. Position Tracking Modules

Examples of the present technology use position tracking modules 102 to determine where the wearable device is within a three-dimensional environment. This positional information can in some examples be combined with hand position information (such as information received from capacitive sensors regarding the positioning of the fingers of the user) to provide a complete picture of the user's hand and fingers within a three-dimensional space.

These position tracking modules 102 can include passive components such as motion tracking markers which enable a camera to track or triangulate the position of the marker within a three-dimensional space. It will be appreciated that where passive tracking markers are used on the wearable device, that a remote device will be required to track the passive tracking marker in three dimensional space.

In other examples the position tracking modules 102 can comprise active components such as inertial motion sensors, accelerometers, gyroscopes, and magnetometers.

In one example of the technology, the position tracking module 102 may be configured to use any commercially available motion tracking or match moving solution. For example, the wearable device may comprise active or passive markers, or may simply have markers of a predetermined size which one or more cameras are configured to track the position and/or rotation of the markers. This tracking and positional determination may be performed using any computer vision techniques known to those skilled in the art, including but not limited to triangulation, machine learning based semantic segmentation and object detection algorithms.

In examples of the technology comprising inertial motion sensors, the position tracking modules 102 may be configured to track movement of the sensors 104 using techniques known to those skilled in the art, such as by detecting rotation with respect to the magnetic field of the earth (in the case of a magnetometer) detecting changes in rotation using a gyroscope and detecting acceleration using an accelerometer. In some examples the position tracking modules 102, may estimate velocity data from acceleration data provided from the one or more inertial sensors by integrating the acceleration data over time. Similarly position/displacement data may be estimated by performing double integration of the acceleration data.

In examples of the technology comprising stress and/or strain sensors such as when using the capacitive sensors described herein. The processor 106 or position tracking module 102 may comprise an electronics module which provides an electronic stimulus such as an oscillating voltage, which allows the impedance, and therefore the capacitance of each of the sensors to be measured. For example, the electronics module may be an application specific integrated circuit configured to measure the capacitance of one or more stress and/or strain sensors.

For any given sensor within the wearable device, it may be possible to define a range of capacitance values which correspond to the sensor being in a loaded or unloaded state respectively. For example, a minimum capacitance value may be representative of a finger at full extension, while a maximum capacitance value may be representative of a finger positioned in a fist, wherein the intermediate positions between the full extension and first may be mapped using any suitable equations.

In some examples of the technology the position tracking module 102 or processor 106 may comprise one or more machine learning algorithms configured to map the sensor 104 readings to positional data as described in greater detail herein.

Further details of capacitive sensing algorithms which may be used with the present technology are described in greater detail in United States Patent Publication No 2020/0319236 A1 the entire contents of which are herein incorporated by reference in their entirety.

### 6.1.2. Sensors

The present technology may comprise any number of sensors configured to provide positional information as described herein. This includes relative positional information, such as hand position information relative to the arm of the user, and/or the relative positioning of any one or more of the user's finger joints with respect to the hand or palm of the hand of the user. For example, the sensors may include cameras, time-of-flight sensors, inertial motion units (IMUs) and stress and strain sensors such as capacitive sensors.

These sensors are preferably operatively connected to the body of a wearable device such as a glove, such that they are able to provide data relating to the location, orientation and/or configuration of the body of the wearable device in use. For example, where stress and strain sensors are used, these stress and strain sensors may be integrated into the body of a wearable device so as to detect the configuration (i.e. deformation) or movement of the body of the wearable device in use.

In some examples of the technology, it can be advantageous to combine multiple sensors to provide detailed positional information. For example, by combining visual information from one or more cameras with relative positional information from inertial motion units, and/or stress and strain sensors, it may be possible to get accurate positioning information which overcomes the shortcomings of each of these technologies individually. For example, inertial motion units, and/or stress and strain sensors may be able to provide positional information while parts of the user are occluded from the view of the camera. Similarly, stress and strain sensors, such as capacitive sensors, when incorporated into a glove may be able to provide accurate repeatable finger position information relative to the palm of the user's hand, which can be difficult to obtain using conventional camera-based imaging techniques.

### 6.1.2.1. Stress/Strain Sensors

Examples of the present technology relate to stress and strain sensors which use soft electronic components in the form of capacitive sensors. Details of suitable capacitive sensors and methods of manufacturing same are described in greater detail in U.S. Pat. Nos. 9,816,800, 10,228,231, 10,539,475 and PCT Publication No. WO2019172781A1 the entire contents of which are herein incorporated by reference in their entirety.

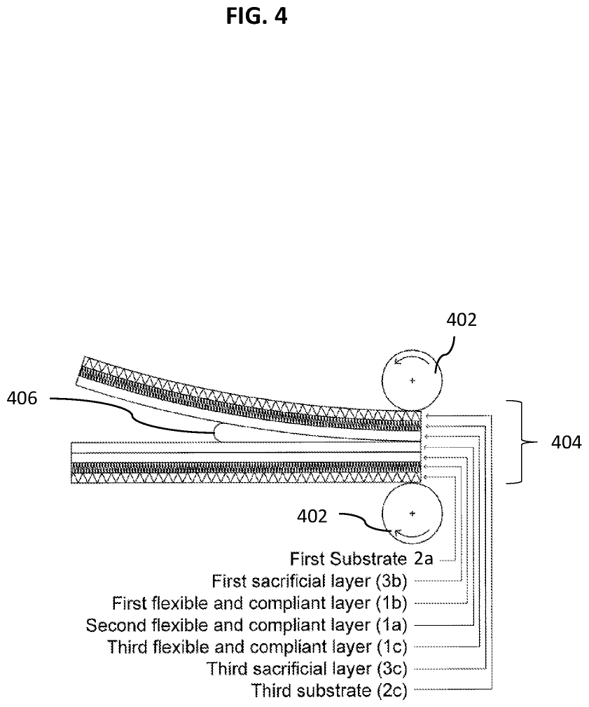

In general terms, the capacitive sensors described herein are laminated sensor devices which comprise two or more electrode films 1a, 1c made from an elastomeric material as illustrated in FIG. 4. The electrode films have a dispersion of conductive particles such that the electrode files are compliant, conductive electrodes. The electrodes 1a, 1c are separated by one or more dielectric film(s) 1b, also made from a compliant or elastomeric material, the dielectric film 1b providing a compliant dielectric layer within the capacitive sensor. This laminated, multi-layer structure 404 acts as a capacitor, whereby the capacitance of the capacitor varies with deformation of the films. This variation in the capacitance can be detected using any capacitance measuring techniques which should be familiar to those skilled in the art, such resonance, or measuring impedance.

In some examples of the technology, the capacitive sensors are formed by providing a first film 1b on a non-compliant substrate 2a, bonding the first film 1b to a second film 1a while the first film 1b is releasably bonded to the substrate 2a, for example to mitigate strain occurring in the first film 1b during bonding. For example, the first film may be releasably bonded to the substrate 2a using a first sacrificial layer, such as an adhesive which can dissolve in the presence of a solvent or water.

Subsequent layers may be formed in a similar manner, for example using second and third substrates, and sacrificial layers 3c. The flexible compliant electrode 1b, 1c, and dielectric layers 1a may be joined together using any technique known to those in the art such as roll forming using one or more rollers 402, press forming, for example using a press (not shown) and/or using an adhesive 406.

Each of the films in the finished capacitive sensor are preferably distinct films, and the action of each of the films from the supporting substrate may laminate the corresponding films together.

In general terms, when incorporated into a wearable device such as a glove, the capacitive sensors can detect movements of the fingers and wrist of the user. For example, when the pose of the hand changes/joints move, stretch is imparted on the sensing zones of the sensors. This causes the electrical properties of the sensors to change, which can include the capacitance, the resistance of the electrodes, and the conductivity of the dielectric, each of which can be affected by stimuli such as deformation as a result of stretching or compression.

In terms of the capacitance measurements, the capacitance of the sensor is proportional to the area of the sensing zone divided by the thickness of the sensor. Accordingly, as the thickness of the sensor changes, such as due to stretching or compression of the di-electric, or as the area of the electrodes change due to stretching, these changes may be detected as a change in capacitance. The position tracking modules 102 described herein can map these changes in capacitance to the amount of stretch imparted to the sensor, and therefore when incorporated in a wearable device, how the stretching correlates to the movements of the user's muscles and joints.

In ordinary operation the sensor operates as an incompressible material. Stretching the sensor therefore causes its area to increase and its thickness to decrease, both of which contribute to an increase in the sensor's capacitance. This particular sensor type is very insensitive to other stimulus, e.g., temperature, time, speed and moisture. Furthermore, capacitance is proportional to the relative dielectric constant of the sensor dielectric, which is silicone and typically has a value of 3 and is has no strong dependency to any stimuli mentioned above. Thus, changes in capacitance are an excellent proxy for changes in the sensor's shape making them excellent deformation sensors.

A further advantage of capacitive sensors is that they are highly repeatable, i.e., if you do the same thing to the sensor, you get the same result. In the context of a glove, moving a joint the same way generates substantially the same sensor signal each time.

This effect can be further enhanced by having multiple sensors placed around the hand. Having multiple sensors to measure multiple degrees of freedom creates a richer description of a particular pose, which makes it easier to resolve more hand poses. Each sensor acts as a coordinate in a multi-dimensional system. Physical hand poses generate a multidimensional array of coordinates that include each sensor value.

Flexible and compliant circuits such as those incorporating a soft capacitor, or other flexible and compliant sensing devices are excellent sensors for soft structures such as the human body. Having a flexible, compliant and lightweight sensing device minimizes any impact on the structure being measured so that it more accurately represents its natural strain or deformation response. As is typical of soft structures, the human body is capable of large, complex movements in 3D space. Digitising large amplitude motion, such as that of the human body, has wide ranging applications in sports, health and fitness, physiotherapy, medical, human machine interface, and entertainment industries, in particular wearable motion capture systems.

The flexible and compliant sensors can be incorporated into a wearable garment, such as a glove, for use in obtaining data relating to the movement of the user. A glove with such sensors may be used to obtain data relating to the movement and position of a user's hand, in particular the fingers. In addition, they are capable of undergoing large stretches with low stiffness so as not to significantly restrict the hand's range of motion.

### 6.1.3. Processors

Throughout the present specification, reference to processors is made in a general sense as being any device or system capable of executing machine instructions. For example, it may include an Application Specific Integrated Circuit (ASIC), a computer processor such as an x86, or x64 processor, and/or a microcontroller.

For example, the processor 106 may be contained within an extended reality headset, or within a personal computing device, such as a smartphone, tablet, desktop or laptop computer. In some examples the processor 106 may be provided within or otherwise attached to a wearable device such as a glove, by including the processor 106 on the wearable device itself it may be possible to process the data provided by the sensors more quickly and reduce the amount of data that needs to be sent between the wearable device and any remote devices.

In some examples the processor is configured to execute computer readable instructions. The computer readable instructions including the steps of receiving sensor data from a plurality of sensors, and either A) communicating the sensor data to an external processor, or B) processing the sensor data to determine hand position data indicative of a pose.

In some examples, the processor may be configured to execute code stored in a memory, such as RAM, flash memory or ROM. In some implementations, the memory may also be a removable or external memory linked to such as an SD card, server, USB flash drive or optical disc, for example. In other implementations, the memory can comprise a combination of external and internal memory. The memory can include stored data and processor control instructions (code) adapted to configure the processor to perform certain tasks, such as the sensor value reading, converting sensor values to finger/hand positions, detecting gestures or poses and/or communicating the information to an external processor.

Where reference is made throughout the present specification to actions or determinations performed by a processor, this should be understood to include common components of computer systems such as a memory containing machine-readable instructions (software/code) which are executed by the process to perform the specified actions. The memory may include any form of non-volatile memory for storing the machine-readable instructions, including but not limited to flash memory, battery powered RAM, read only memory (such as electrically erasable programmable read-only memory EEPROM), as well as volatile memory such as RAM. These components are typically provided on a printed circuit board assembly (PCBA) as should be familiar to those skilled in the art.

In some examples of the technology, where the processor is configured to process data provided by the sensors in use, this data may be stored in volatile memory such as RAM, until processed, and the resulting hand position information may similarly be stored in volatile or non-volatile memory. For example, in some examples it may be beneficial to keep a record of hand position information for downloading or transferring to a remote device at a later point in time, such as at the end of a motion capture recording session. In other examples, it may be beneficial for the hand position information to be stored temporarily, for example for near instantaneous transmission to a remote device via a communications module, or for further processing. This example may advantageously result in a lower cost device, and/or a device which is simpler to use.

The processing capabilities of the processor may be provided, for example, by one or more general-purpose processors, one or more special-purpose processors, or cloud computing services providing access to a shared pool of computing resources configured in accordance with desired characteristics, service models, and deployment models.

Although the processor and memory described herein are described in the context of being single unit(s), it should be appreciated that this is not intended to be limiting, and that the functionality of each as herein described may be performed by multiple processors and memories, that may or may not be remote from each other and the remainder of system.

### 6.1.3.1. Machine Learning

Certain aspects of the present technology may take advantage of machine learning technologies in order to perform any one or more of:
Processing sensor data to determine hand position information;
Determining the likely hand parameters of the user such as the size and shape of the user's hand in order to account for user-to-user variation in the sensor data;
Processing the hand position information to determine whether the hand position information corresponds to a pre-configured set of hand positions linked to one or more events; and
Determining when commands should be sent to a remote device.

For example, a model trained by machine learning may be used on the wearable device to process information captured by the wearable device (such as by the sensors on the wearable device), or on a remote device communicatively connected to the wearable device to process information sent from the wearable device to the remote device.

For example, the wearable device may comprise a memory containing machine-readable instructions in the form of a model trained by a machine learning algorithm. These machine-readable instructions may then be executed by a processor on the wearable device to perform any one or more of the actions described above.

In one example of the technology, a machine learning model configured to determine hand position information from sensor information may be trained by:
A) Equipping a hand with a wearable device in the form of a glove;
B) Manipulating the hand into a predetermined position; and
C) Acquiring capacitance values from the wearable device.
D) Using the hand position information and the capacitance information as labelled training data for a supervised learning model.

The above steps may be repeated for a range of different hand positions, with a range of different users, and glove sizes and configurations. This approach can advantageously allow the model to determine accurate hand position information irrespective of the physical properties of the user's hand. Accordingly, a database of capacitance values may be obtained which can be mapped to various hand positions.

In some examples of the technology, the hand may be an electromechanical hand, wherein the angles and positions of each of the fingers may be acquired digitally. In other examples of the technology the hand may be the hand of a user, and the user may be instructed to manipulate their hand into a predetermined position.

In other examples of the technology, the hand may be positioned in the viewport of one or more cameras configured to monitor the position and orientation of the fingers of the user. Accordingly, in this example, the user may move their hand through a range of motion, and the corresponding finger and hand positions recorded by the camera and mapped to the corresponding capacitance values provided by the wearable device. In other words, the combination of sensor information and hand position information obtained by the camera can provide a database of labelled training data which allows one or more models to be trained for each user hand size.

Accordingly, in the above examples the models may be used to determine hand position information from the sensor information. However, in some examples of the technology, it may also be beneficial to store a pre-configured set of hand positions and train an AI model to determine whether the user has performed one or more of the pre-configured set of hand positions. These pre-configured hand positions can in some examples be linked to events that the user intends to perform as described herein, such as controlling a mode of operation of the wearable device or sending commands to a remote device.

Accordingly, another aspect of the technology is to use machine learning techniques to train a machine model to determine how closely the hand position information matches one or more pre-configured hand positions. For example, using the techniques described above, a user wearing a wearable device may be instructed to perform a pre-determined hand pose (such as the thumb up pose for example). The processor will as a result convert the corresponding sensor data to hand position data, such that there is linked sensor data, hand position data and pose information which can be linked to each other for supervised learning of the machine learning model.

As in the previous examples, it may be beneficial to train the model on a wide range of user hands to increase the tolerance of the model to variations in specific user hand parameters.

When multiple pre-determined hand positions are provided, the model may be configured to create boundaries in the multidimensional system and to output the pre-configured hand position (pose) the user's hand is closest to from the trained poses. In this example, there is no interstitial space between trained poses, so the output is guaranteed to be one of the trained poses. This is particularly useful when there are only a specific number of desired outputs, and any other poses are not relevant/necessary. This mechanism may be used for example as a filter.

This enables the system to interpolate between the boundaries of the range of motion that is captured during the training process. For example, by training the system with two static poses: a flat hand and a fist, the system can credibly predict intermediate stages where the fingers are bent by taking the instantaneous raw sensor values and calculating a weighted average of each reference pose. Alternatively, a regression algorithm may be used to define a line of best fit to the data that represents the relationship between the raw sensor values and the position within the range of motion.

This can be further enhanced by breaking the raw sensor data into subsets and focusing on, for example individual fingers so that the training data can be used to predict the position of each degree of freedom independently of other degrees of freedom. In a glove application this enables fingers to be predicted independently, e.g., the index finger can be half curled while the other fingers are straight.

As well as selecting the closest output pose, it is possible to obtain a confidence score that provides an indicator as to how close the wearer's hand is to the closest known pose. For example, this can be mathematically derived from the instantaneous raw sensor values when compared to the sensor values captured during the training process. i.e., where the average sensor values correspond more strongly to a first pose than a second pose, the system may be able to provide a confidence score which is indicative of the percentage difference between the expected pose values and the observed pose values. It should be appreciated that these confidence scores may be provided for the gesture generally, or for any one or more of the sensors within the wearable device, such as per finger, or per degree of freedom being monitored by the sensors described herein.

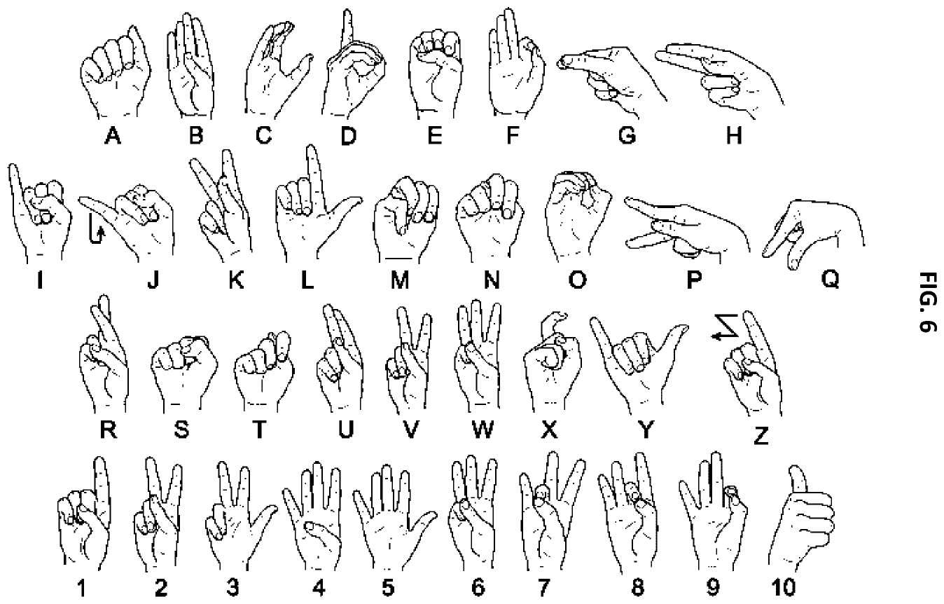

Thus, it is possible to add an additional layer of fidelity in which for example you could output a default state if the confidence score for the most likely pose is not above a user defined threshold. In other examples, the processor may be configured to send a command to a remote device, only when the confidence score exceeds a predetermined threshold i.e., only when the confidence of detecting a pre-configured hand position exceeds a certain threshold. This threshold may be set for all pre-configured hand positions, i.e., such as being greater than 80%, or may be set per pre-configured hand position. For example, the first pose may be relatively easy to detect since it results in relatively large displacement in all sensor values, and therefore this may be detectable with a high level of confidence such as greater than or equal to 80%. Conversely, when determining whether a user is performing the American sign language (ASL) pose for the letter K versus the letter V (as illustrated in FIG. 6), the confidence of classification may be lower, and therefore the processor may be configured to send a command to a remote device depending on which classification is greater (even in the event that both confidence scores are high such as greater than or equal 80%).

Similarly, some preconfigured hand positions may be more difficult to detect, for example due to variations in sensor values between users, and therefore in these situations it may be preferable for the processor to send a command to a remote device when the confidence score for that predetermined hand position exceeds a relatively low threshold such as greater than or equal to 60%.

The inventors have found that it is critically important for pose reproduction to work that consistent training data is used to train the machine learning model. That is, where physical joints are in the same position between two different poses, it is essential that they are also in the same position in the reference data.

Whether using the glove and algorithm to detect the nearest applicable pre-configured hand position (pose), which may or may not be required to meet a certain threshold/be within a desired proximity to the pre-configured hand position, or whether a weighted average or regression model of all training information is desired, this process is highly flexible. The specific array of sensor data that is achieved by the wearer posing their hand in the desired pre-configured hand position can be correlated to a specific output. The user can arbitrarily define the desired state they want the system to enter into upon recognizing a pose and use either the state itself or the change of state to trigger specific actions or events.

Accordingly, the machine learning algorithm can be trained to recognize one or more preconfigured hand positions including but not limited to: a closed fist, an open hand with all fingers touching, and open hand with all fingers separated, a closed first with any combination of fingers extended, and any combination of poses therebetween. Examples of preconfigured hand positions include for examples the American Sign Language (ASL) hand positions corresponding to the letters of the alphabet as illustrated in FIG. 6.

In some examples of the technology, it may be advantageous to include one or more sensors mounted on or adjacent to the wrist in order to determine or infer the position or orientation of the wrist in use. For example, an accelerometer mounted to the wearable device adjacent to the hand or wrist may be able to detect a downward direction (i.e., inferior direction, or direction facing towards the ground), due to acceleration under the force of gravity. Accordingly, orientation relative to the downward direction may be determined by the axis of the accelerometer which receives values indicative of the gravitational force.

In other examples of the technology, the one or more sensors may comprise, capacitive sensors configured to detect the wrist twisting or bending therefore changing the position of the user's hand with respect to the user's forearm or elbow.

Taking a snapshot of every sensor value simultaneously defines a location in a multi-dimensional system that incorporates at least the individual sensor values as coordinates in this system that can be correlated with an arbitrary reference output of the user's choice. This can be correlated with the matching pose of a virtual hand to create a real-time digital reproduction of the user's hand, for example by using the machine learning model described herein, or alternatively it may be correlated with an event name or similar definition that can be used to trigger further actions.

Where hand poses are correlated with a digital twin of the hand, the machine learning algorithm can be trained using all of the reference data.

### 6.1.3.2. Digital Hand Modelling

One method for visualising in real time the detected behaviour of the user's hand is to generate a digital copy (often referred to as a digital twin) of the user's hand. This digital hand may be presented to the user, such that they can see the orientation and/or position of the hand changing in real-time or near real-time.

Accordingly, for any given capacitance value, or combination of capacitance values, it is possible to determine hand position data which includes a likely position for each of the fingers within the wearable device. This hand position data can be presented as a virtual model, for example by using defined minimum and maximum capacitance values described herein, or alternatively using one or more machine learning trained model as described above.

A copy of this virtual model may be used by the processor 106 on the wearable device in order to determine whether the hand position information matches on or more pre-configured hand positions as described herein, and in some applications, such as motion capture it may be beneficial for the virtual model to be communicated to at least one remote device, in order to allow for visualisation of the hand in near-real time. For example, the virtual model may be communicated as finger and hand joint angles communicated periodically via the communications module. The frequency of communication may depend on the intended application. For example in applications requiring high precision this information may be communicated 50 times per second or greater, while in other applications, it may be sufficient to send the information less frequently to save on power, such as approximately 10 times per second.

### 6.1.4. Wearable Devices

The present technology is directed to tracking technologies that may be used to track the anatomy of a body, such as a human body. The concepts described herein are generally applicable to any part of the anatomy, however for sake of simplicity the examples used herein describe uses of the technology for hand position tracking and control systems. This however should not be seen as limiting on the technology, for example the present technology may be used in other wearable devices such as socks, shoes, compression tights, shirts, chest straps, sweat bands, and other articles of clothing.

A first example of a wearable device 306 is illustrated in FIGS. 3D and 3E, in this example the wearable device 306 is provided as a glove. The use of gloves should not be seen as limiting on the present technology and is described herein as being one of the most complex shapes on the human body to track due to the large number of joints and the degrees of freedom of each. Accordingly, as the technology is proven to work in the glove application, it may also be applied to less complex parts of the human anatomy without departing from the spirit and scope of the invention. For example in some examples the technology may be used to track the movement of a user's feet and ankles, for example by being integrated into socks. In another example, the technology may be used to track the body, for example by being integrated into a shirt, or similar clothing. Similarly, the present technology may be used for non-human applications such as monitoring and/or obtaining digital motion capture for animals such as dogs, cats or horses etc.

Gloves in general should be familiar to the general public, however for sake of completeness, the glove comprises a body 302, which is configured to receive the user's hand in use. The body of the glove may in some examples of the technology include separate finger compartments 322 configured to receive each of the fingers of the user. These finger compartments 322, can in some examples be open ended to provide a “fingerless glove” as should be familiar to those skilled in the art. In contrast, where the wearable device is a sock, the corresponding features of the body still apply, and the corresponding discussion may be in relation to the toes and or feet of the user.

In use a plurality of sensors 104 may be attached to the body, such that deformation of the body (of the glove or sock for example) causes a detectable change in the sensors 104. For example, capacitive sensors may be used as described herein, which allow for strain to be measured at locations in the glove which correspond to locations on the user's hand.

In preferred examples of the technology, the plurality of sensors may be provided at locations which correspond to one or more joints and/or degrees of freedom for each of the user's fingers, and wrist. For example, for each finger, a first sensor 104A may be provided adjacent to the metacarpophalangeal joints and/or scapho-trapezium/scapho-trapezoid joints, a second sensor 104B may be provided adjacent to the proximal interphalangeal joint, and a third sensor 104C may be provided adjacent to the distal interphalangeal joint. In this way transitioning the user's hand from a flat extended position to a closed, first position may cause a stretch in the sensors, causing deformation that may be measurable by the processor 106 or position tracking modules 102 described herein. For example, each of the first, second, and third sensors described above may be mounted to a rear of the glove, such that they are proximal to the corresponding joints of the user on the rear of the user's hand.

In examples of the technology comprising fingerless gloves, the third sensors 104C may be ommitted, and data from the first 104A and second sensors 104B may be used to estimate the angle of the distal interphalangeal joint, as for most users, the angle of this joint directly corresponds to the angle of the proximal interphalangeal joint.

In the case of the thumb of the user, it should be appreciated that there is only a single interphalangeal joint, and as such for the sake of this discussion, this has been designated as a second sensor 104B, although this may be omitted in examples of the technology where fingerless gloves are used. In addition, the first sensor 104A when used in relation to the thumb may further be configured to detect movement or deformation in the user's scapho-trapezium/scapho-trapezoid joints. Thereby potentially providing more accurate tracking of the user's hand position information in comparison to wearable devices which do not actively track the position of these joints.

In some examples of the technology, for example where it may be desirable to detect the splay, or spreading of one or more of the fingers, it may be advantageous for one or more sensors (herein referred to as fourth sensors 104D) to be positioned on the positioned on or adjacent to the sides of the metacarpophalangeal joints (knuckles). In this way, as the finger moves laterally, relative to the palm of the user's hand, within the plane of the user's hand this movement may be detectable as a compression or extension of the corresponding fourth sensors 104D.

In other examples of the technology, the glove may comprise one or more sensors 104 (herein referred to as fifth sensors 104E) positioned on or adjacent to the wrist joints of the user, such as above the radiocarpal joint (i.e., on the back of the user's wrist) on the inside of the user's wrist, and/or to the sides of the radiocarpal joint, such as adjacent to the radial or ulnar zones on the sides of the wrist.

In use each of the sensors 104 in the wearable device 306 are operatively connected to a position tracking module 102. for example, the position tracking module may be configured to perform measurements on the sensors, to obtain one or more parameter of the sensors, such as the capacitance of the sensor 102. In some examples of the technology the position tracking module 102 may further convert this capacitance information to positional information in the form of joint angles, or finger position information, however this should not be seen as limiting, and in other examples the processor 106, may be configured to determine the positional information.

In some examples of the technology the wearable device 306 may comprise an electronics housing 310 as illustrated in FIG. 3F configured to contain one or more of: a position tracking module 102, a processor 106, a power source 312, and a communications module 314, such as a wireless communications module or Bluetooth™ module.

For examples the communications module 314 may be operatively connected to the processor (wirelessly or via one or more wires) in order to receive information from the processor, and communicate that information with one or more remote devices. In some examples the communications module 314 may be configured to receive information from a remote device, such as context information as described herein.

For example, the electronics housing 310 may be a substantially rigid plastic housing provided with a watertight seal to prevent water ingress which may otherwise damage the components (such as the processor 106, power source 312, communications module 314 etc.) contained therein.

The processor 106 and/or position tracking module 102, may be configured to communicate with the one or more sensors, for example using an electrical interconnect 316, such as conductive trace on a flexible printed circuit, or using a wire. Method of connecting capacitive sensors to sensing electronics are described in greater detail in PCT Publication No. WO 2019/022619, the entire contents of which are herein incorporated by reference in their entirety.

The body 320 of the glove may in some examples be formed from a single layer of material, or a multilayered material structure. For example, the material may comprise any one or more of: fabrics such as cotton, polyester, linen, animal skins such as hides and leather (including synthetic leathers), polymers such as nitrile, latex, polyurethane, and fibre materials such as Kevlar.

In examples of the technology, where the body 320 is comprised of a single layer of material, the sensors 104 may be mounted, preferably on non-user contacting side of the glove, adjacent to the rear or back of the user's hand. In other examples, the sensors 104 may be mounted on a patient contacting side of the glove, and/or adjacent to the inside/palm of the user's hand.

In some examples of the technology, the body of the glove may be constructed, from a plurality of material layers, for example a user contacting layer, configured to engage with the skin of the user, and a non-user contacting layer, facing outwardly from the skin of the user.

In some examples the non-user contacting layer may be provided with a grip in one or more regions of the glove, such as in the palm and or inner side of the fingers of the user. The use of a grip may advantageously assist the user in gripping objects while using the wearable devices described herein.

In some examples the non-user contacting layer may be provided with one or more active or passive markers configured to be detected by a camera in use. For example, the active or passive markers may be provided on the region of the hand adjacent to the wrist of the user, the back of the user's hand, and/or the sides of the user's wrist. The use of active or passive markers may advantageously reduce the processing burden on camera tracking systems as described herein.

In some examples of the technology, the sensors 104 may be positioned between the user contacting layer, and the non-user contacting layer, for example the sensors may be mounted to a non-user contacting side of the non-user contacting layer, using any techniques known to those skilled in the art, including thermoforming, welding, stitching and using adhesives.

### 6.1.5. Other Devices

While the foregoing discussion primarily focuses on wearable devices configured to detect movement in a user's hands (i.e., gloves) this should not be seen as limiting on the technology. For example, as described herein, the technology may be applied to any garment, such as socks, shirts, shoes, headbands, wristbands, knee, shoulder and wrist braces etc.

Reference herein to a user, should also not be seen as limiting for example, in some cases the user may be an animal, or a robot, as opposed to the more conventional human applications.

Accordingly, in a general sense the wearable devices 306 described herein comprise a body which comprises a user contacting side, configured to contact a user in use. In examples, the body comprises one or sensors configured to detect movement of the user, via deformation of the one or more sensors. In some examples of the technology, the wearable device 306 comprises a position tracking module 102, configured to receive information from the one or more sensors, and optionally a processor configured to trigger one or more actions based on the movement detected wearable device.

### 6.1.6. Processing Systems

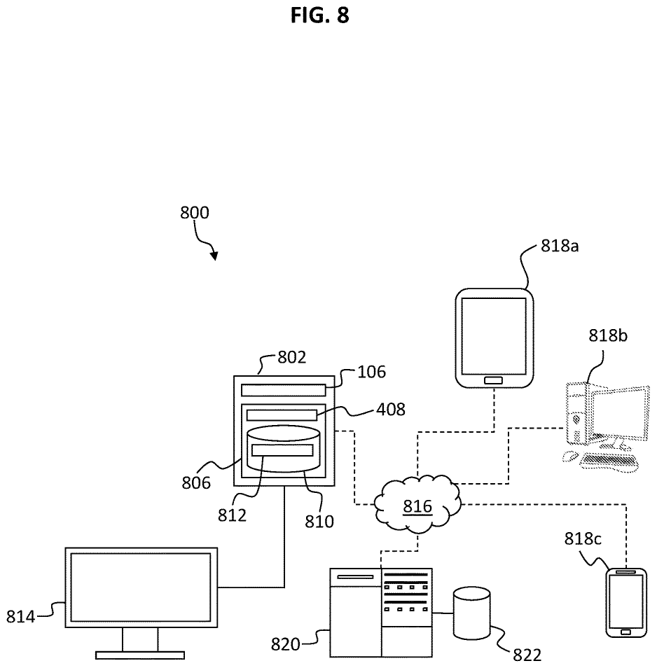

FIG. 8 is a schematic illustration of an exemplary processing system 800 according to one form of the technology. The processing system 800 may comprise a hardware platform 802 that manages the collection and processing of data from the wearable devices 306 described herein, such as the data received from one or more sensors, and/or the hand position data. The hardware platform 802 may comprise a processor 106, memory 806, and other components typically present in such computing devices. The hardware platform 802 may be local to the wearable device 306 or it may be remote from the wearable device 306 and receive the data over a suitable communications link, such as network 816. In the exemplary form of the technology illustrated, the memory 806 stores information accessible by processor 106, the information including instructions 808 that may be executed by the processor 106 and data 810 that may be retrieved, manipulated, or stored by the processor 106. The memory 806 may be of any suitable means known in the art, capable of storing information in a manner accessible by the processor 106, including a computer-readable medium, or other medium that stores data that may be read with the aid of an electronic device.

The processor 106 may be any suitable device known to a person skilled in the art. Although the processor 106 and memory 806 are illustrated as being within a single unit, it should be appreciated that this is not intended to be limiting, and that the functionality of each as herein described may be performed by multiple processors and memories, that may or may not be remote from each other or from the processing system 800. The instructions 808 may include any set of instructions suitable for execution by the processor 106. For example, the instructions 808 may be stored as computer code on the computer-readable medium. The instructions may be stored in any suitable computer language or format. Data 810 may be retrieved, stored or modified by processor 106 in accordance with the instructions 810. The data 810 may also be formatted in any suitable computer readable format. Again, while the data is illustrated as being contained at a single location, it should be appreciated that this is not intended to be limiting and the data may be stored in multiple memories or locations. The data 810 may also include a record 812 of control routines for aspects of the system 800.

The hardware platform 802 may communicate with a display device 814 to display the results of analysing the data. The hardware platform 802 may communicate over a network 816 with one or more other devices (for example user devices, such as a tablet computer 818a, a personal computer 818b, or a smartphone 818c, or other devices including sensors), or one or more server devices 820 having associated memory 822 for the storage and processing of data collected by the local hardware platform 802. It should be appreciated that the server 820 and memory 822 may take any suitable form known in the art, for example a “cloud-based” distributed server architecture. The network 816 may comprise various configurations and protocols including the Internet, intranets, virtual private networks, wide area networks, local networks, private networks using communication protocols proprietary to one or more companies, whether wired or wireless, or a combination thereof.

In certain forms, the hardware platform 802 of data analysis system 800 may comprise a computing device, for example a laptop or PC. In other forms, the hardware platform 802 may comprise a plurality of computing devices configured to operate collectively to perform the data analysis/processing.

### 6.2. Gesture/Pose Detection

### 6.2.1. Definable Conditions

Throughout the present specification reference is made to hand position information, gestures and poses. In general terms poses and gestures both fall under the broader category of hand position information, however a pose refers to a static hand position, while a gesture includes movement, whether it be movement into or out of a pose, or movement when in a pose (i.e., waving generally involves a fixed, hand open pose, with all fingers extended, with the gesture movement provided by wrist or arm movement).

The use of gesture and/or pose detection can be useful for ensuring that intended behaviours are performed. For example, in a game, a user may be able to pull out and aim a firearm by making a gun pose with their hand, i.e., pointer finger extended, middle, ring and pinkie fingers curled into a fist. However, it may be advantageous to ensure that the action of pointing the user's finger only pulls out the firearm when the user intends for it to do so. For example, it may not be desirable to draw the firearm for example when performing a similar gesture to press an elevator call button for example.

Similar examples can include locomotion within virtual environments, this is typically a behaviour which can be disorienting and result in motion sickness if implemented poorly or performed an unexpected times. Therefore, in some examples it can be important to ensure that the processor 106 is aware of the context or intent of the user, before performing one or more actions or sending one or more commands.

Accordingly, one aspect of the invention is to provide definable conditions under which a pose or gesture triggers a corresponding action. For example, these conditions may be user definable in software configured to execute on the processor 106 or position tracking modules 102 described herein, in other examples the conditions may be definable in the wearable device, such that the corresponding action is only sent to the processor if the conditions are met.

The conditions may comprise any one or more of:
A defined duration for the pose or gesture. For example, the user may need to hold the preconfigured hand position/pose for a defined duration before the corresponding action is triggered, in other words a minimum duration condition may be set. Alternatively, a maximum duration may be set, for example the condition may require that the gestures are performed at a minimum speed before the action is triggered.
A sequence of poses or gestures, for example the condition may require a first pose, followed by a second pose before an action is triggered. Alternatively, the condition may require that the gesture involves a transition from a first pose to a second pose.
Context sensitivity. For example, a condition may be set such that an action is only triggered if the user is adjacent to (such as within a defined distance of) one or more virtual objects in a virtual environment.
One or more external inputs. For example, the condition may require a button press on one or more handheld controllers 120 before the action is triggered.

### 6.2.2. Triggered Actions

Depending on the use case for the present technology, the detected gestures and poses may be used to trigger or affect any suitable action for example by sending a command to a remote device. For example, this may include (but is in no way limited to):
Starting, stopping, or pausing an application.
Opening, closing, or navigating a menu.
Interacting with a virtual object such as moving, scaling, transforming, rotating, editing the mesh of the object, rendering, texturing, or painting.
Sending one or more key presses or mouse inputs.
Sending one or more multimedia control signals, such as volume control, or pitch adjustment.
Interfacing with one or more home automation systems to control the operation of a real-world device, such as turning a light on or off, adjusting brightness or tone, turning an appliance on or off, adjusting the temperature or fan speed of an air conditioning unit, opening or closing a door (such as a garage door) etc.

One application of the technology is to mimic existing control schemes for applications. For example a game may be configured to receive controller inputs, and as such in one example the triggered action may be to send one or more controller inputs to the game/application. For example when the processor 106 detects hand position information corresponding to a trigger pull pose or gesture, the processor may be configured to send a trigger press, such as an “R1” or “R2” trigger press which would be mapped to a shooting action in game.

In another example, the user may perform a grabbing pose or gesture which mimics a user grabbing a joystick or steering wheel. This can provide the processor with context information that the user intends to pilot/drive a vehicle. Accordingly the processor 106 may use this context information to provides accelerometer data from an on board IMU, which mimics the hand position, such that tilting, or rotating the user's hand sends corresponding joystick X and Y directions to the remote device. Without this context the processor may be configured to send no information, or instead send a different command (for example relating to a different context).

Accordingly, the commands described herein can include any one or more of: keypresses; multi-media commands (such as pause, play, stop, fast forward, volume up/down, brightness up/down, mute etc); displacement on one more axes (i.e., joystick or mouse positional information); finger position data; or hand position data. In some examples the data used for these commands may be obtained from an inertial motion unit, accelerometer, gyroscope or magnetometer. For example the axial displacement may be obtained from an orientation of the wearable device.

### 6.2.3. Context Switching

In certain examples of the technology, it may be advantageous for the gesture and pose detection systems described herein to allow for context switching between different applications, or different functions within an application running on the processor 106.

For example, in three-dimensional modelling software it may be advantageous to switch between a rendered view, a wireframe view, or a solid, unrendered view. Similarly, it may be advantageous for the present technology to allow for switching between a vertex/face/object selection function, and a vertex/face/object editing function.

Other examples include a game, in which it may be advantageous to switch between a movement function and an interaction function. Or in a remote surgery application it may be advantageous to switch between a robotic arm movement function, and an inspection function.

Accordingly, by using a specific gesture, or pose, or a sequence thereof it may be possible to provide the processor with appropriate context information which controls or otherwise modifies the commands send to the remote device to ensure that they are appropriate to the context.

### 6.2.4. Mode Switching

It can be beneficial for wearable devices to provide multiple functions depending on the intended application or use case. This can be achieved for example by using different Bluetooth profiles for the intended use case.

For example when performing a software update, or transferring information it may be advantageous for the wearable device to present itself as removable media. In other examples, such as controlling a game, it may be advantageous for the device to present itself as a game controller, or a generic human interface device such as a keyboard or mouse.

Similar behaviour can be observed in modern smart phones where upon connection to a computer, a prompt is issued as to whether the user intends to charge the device, transfer files, transfer photos, enter a debugging mode etc.

However when these modes are switched it can result in a disconnection and reconnection, which adds latency and is an inconvenient user experience. Furthermore, while this interface makes sense on a smartphone, it is often inconvenient to implement similar functionality on devices that do not have a display (such as wearable devices as described herein).

Accordingly, one aspect of the technology is the ability for the processor to switch between operating modes based on information provided by a remote device, or by the user performing a pose or gesture which corresponds to a pre-configured pose or gesture which has been set specifically for changing the mode. In some cases, it may be advantageous to require a sequence of pre-configured poses or gestures before switching modes to thereby reduce the likelihood of unintended activation/switching, however this should not be seen as limiting.

It should be appreciated that mode switching in some cases may result in the wearable device reconnecting as a different peripheral, for example switching between a first mode in which the wearable device is emulating a controller or keyboard, (for example to interact with a console or pc) and a second mode in which the wearable device is in a data transfer mode (for example to send hand position data for motion capture applications). In some examples the first mode and second mode may be active simultaneously, i.e., sending both data streams simultaneously.

In other examples, the mode switching may be performed without reconnecting as a different peripheral. For example, depending on desired applications, it may be possible to communicate data in a first mode, using the same protocols as data in the second mode. For example the first mode may comprise hand position data, and the second mode may comprise commands, both of which are communicated using a generic human interface device profile. In this example the hand position data may be provided by a steam of alphanumeric key presses possible on a keyboard, while the commands may comprise mouse clicks, mouse movements, use of the function keys, multimedia controls etc.

### 6.2.5. Gesture/Pose Detection

Gestures and poses may be defined using any of the techniques described herein. For example, these may include predefined gestures and poses, learnt through a machine learning training model, whereby the user is asked to perform a pose or gesture, and the learning algorithm assigns the positional information measured to the corresponding gesture or pose. In another example, a user may manipulate a virtual hand using software, and when the virtual hand is in the desired pose, a corresponding action may be assigned. Similarly, gestures may be defined, either by performing the action in a wearable device once prompted, or by defining two poses, and the gesture as being the action which is taken to transition from the first pose to the second pose.

When multiple poses and/or gestures trained or learnt by the systems described herein, it is possible to compare that pose or gesture against a plurality of trained poses and/or gestures to determine how closely the reference pose or gesture matches the learnt pose or gesture. For example, a confidence score may be generated using a mathematical comparison of the sensor values or tracked finger/hand positions against the learnt sensor values/tracked finger/hand positions. For example, for a match, the system may require that all sensor values, or hand/finger positions are within a certain percentage of the learnt sensor values or hand/finger positions, such as within 10% or within 5%.

The use of wearable devices as described herein may be particularly advantageous, for example the use of capacitive sensors may allow the positional sensing to be both reliable and repeatable. In other words, if you do the same thing to the sensor then you receive the same result. The measurements do not suffer from occlusion issues present in camera systems, or drift issues present in inertial measurement systems.

### 6.2.6. Linked Gestures/Poses

In some aspects of the technology, certain gestures or poses may be connected to other gestures/poses or contextual information that can be used to modify or adapt the output of the system dependent on any one or more of:
the order of poses;
the position and orientation in space of the hand; and/or
proximity to an interactable object for example.

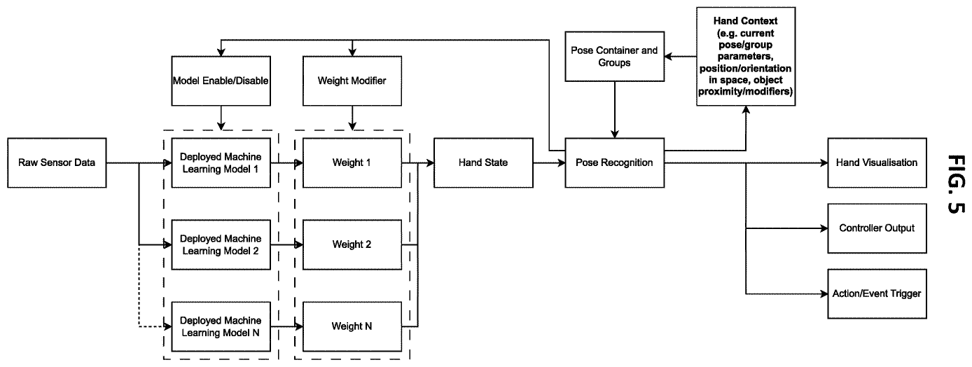

For the sake of brevity, we will refer to poses in the following example but the same applies to gestures. In the present invention poses can be organised into a hierarchy of: pose containers, pose groups, and poses as discussed in relation to FIG. 5.

In the case of a wearable device such as a glove, a pose container represents all of the different poses that can be detected from that hand for a given context. Active containers may be swapped on and off the hand based on context. For example, by default a hand may have a set of generic poses for grabbing virtual objects or for activating features such as raycasting, for interacting with far off objects, or teleporting the user around a virtual world when they do not have sufficient physical space in the real world to locomote on a 1:1 basis.

In one example, a specific virtual object may have its own pose container that is to take precedence when the hand is in proximity to the object. For example, a virtual coffee cup may have several ways specific to the dimensions of the cup of how it can be picked up, e.g. using thumb, index and middle finger to grasp the handle, holding the side of the cup directly, pinching the rim of the cup between thumb and index, cupping the cup from underneath. This information is made part of the meta data of the cup itself, or attached to the object for example as a script or piece of code. Accordingly, when the hand is in proximity to the virtual cup this information may be used to determine which of the poses in the pose container is closest to the pose being performed by the user.

Accordingly, by using proximity to one or more virtual objects it may be possible to limit the total number of potential poses or gestures that would make sense in that application. Accordingly, when the aforementioned system is determining which pose or gesture the user is performing, information relevant to the virtual object may be used to override the default pose container that the hand already has. This method reduces the number of poses that need to be evaluated at any point in time due to its contextual sensitivity and subsequently greatly increases the diversity of poses that can be recognised without risk of a conflict. This is a powerful tool for highly computationally efficient methods for quickly adapting the pose of the hand to the specific shape of an object.

In one example of the technology, a pose container consists of pose groups. A pose group represents poses that are closely coupled. For example, pose groups may have 4 main properties:
1) Are the poses in the group selectable if the hand is not near an object,
2) Are the poses in the group sequenced,
3) Is the group locked, and
4) The list of poses themselves.

If a pose is selectable in free space it can be activated when the hand is not in proximity to a virtual object, whereas if it is not selectable in free space then the hand must be in proximity to a virtual object for the pose to be evaluated. A pose group that is sequenced means that poses can only be activated in the predefined sequence, e.g., the only way to get to the second pose is if the first pose is executed first. If it is not sequenced, then any pose in the group can be activated from some starting point. For example, a typical method of selecting far away objects in virtual environments is the use of ray casting or a virtual laser pointer where a user must activate the ray cast, target the desired interactive object, and perform a confirmatory action to activate the selected object. In this case it is beneficial to the user to a sequenced group with a recast start pose (e.g. a finger gun with index finger and thumb extended and middle, ring and pinkie fingers curled into a fist), and a ray cast activate pose (e.g. the same pose with the index finger curled as if to pull a trigger).

By making the poses distinct and in a sequence, it greatly decreases the risk of false positives, and prevents the user from inadvertently activating a far off object by going to the activated state directly. A group of poses may also be group locked, i.e., once one pose in the group is activated, the next pose can only be another pose that belongs to the same group, or not group locked, meaning that when that pose is activated it is possible to jump directly to any pose in a non-sequenced group or the first pose in any other sequenced group. Again, this contextual sensitivity enables a wider variety of poses to be recognised more reliably as compared to a configuration where all poses belong to one group for example.

The definitions of poses themselves comprise of three main components, with room to provide additional optional components. The first main component is the activation threshold for the pose. This is the criteria the hand shape must meet in order for the pose to be activated. In this example we will use the normalised range from 0 to 1 representing the full range of motion of a particular degree of freedom in the hand (0 typically being straight/not bent and 1 being maximally rotated along the degree of freedom) but the same principle applies if different mathematical representations of the hands are used. In defining the activation criteria the first parameter is the comparison logic which in the simplest case is AND (all of the subsequent criteria must be met for activation) or OR (activation occurs if any of the criteria are met), although it will be apparent that this can be naturally extended to other logical combinations, or combinations of combinations. In some examples of the technology, for activating a pose, AND logic is used. 1 or more degrees of freedom may be evaluated in the activation test, and for each degree of freedom that is considered, the developer can specify a minimum and a maximum range that that degree of freedom must sit within to be considered active. Depending on the selected logic, if the criteria are satisfied the pose will activate.

The second main component is the holding thresholds for the pose once it is activated. The first parameter is the comparison logic, which can be AND or OR. The user, or a software developer can then specify a minimum and a maximum value of 1 or more degrees of freedom that that degree of freedom must remain within to satisfy that criteria. Typically, the comparison logic used in the holding thresholds is OR. That is, if any of the degrees of freedom of interest fall outside of the range specified in the criteria, the pose will deactivate. Critically to the function of this feature, the activation criteria and the holding criteria can be set independently. This ensures activation and holding thresholds can be set that prevent ringing or bouncing of poses if the hand is on the cusp of activation.

The final main component of the pose definitions is the degree of freedom constraints. When a pose is active, it is possible to impose temporary limitations on the range of motion of a particular degree of freedom to facilitate consistency of what the virtual hand looks like when it is interacting with a virtual object. This can be in the form of clamping, that is where a degree of freedom may normally range between 0 and 1, while when a particular grab is active this may be limited to 0 to 0.5, or scaling, where a scalar or other transformation function is applied to the raw value of the degree of freedom before it is applied to the hand animation. For example, clamping is useful when you are holding an object such as a ball, whereby limiting the maximum curl of the fingers to stop at the surface of the ball, while still allowing the fingers to be lifted off the ball, it provides a highly computationally efficient method for ensuring the hand holding the ball looks natural without being completely wooden without relying on heavier physical simulation calculations.

It will be appreciated in the context of the linked poses and gestures the primary goal is not limited in scope to being the visualisation of the virtual hand, although it is very powerful in this regard. Specific poses or pose sequences that the user can deliberately and reliably activate or execute can be used to trigger any events, not just control the appearance of the hand. In particular, activation poses can be linked to switching between different models for processing the raw data coming from the wearable device/glove.

For example, in the present invention detecting the region of the range of motion the hand is in is used to control the relative weighting of the output of multiple machine learning models, up to and including switching some models off and switching other models on after a particular pose has been activated and whilst it is satisfying the holding criteria. In the present invention, finger splay is more accurately predicted by a machine learning model trained on just splay data when the fingers are nominally straight (a fingers ability to splay diminishes as the fingers bend) and running it in parallel with a general model for predicting finger position and using pose estimation to increase the weighting of the splay model output when the fingers are near straight.

Taken to a more extreme level, poses, which themselves can be sensitive to whether the hand is interacting with a virtual object or not, can trigger certain model outputs to be switched on and other model outputs to be switched off to modify the overall output to have a narrower scope with higher fidelity within the range of motion. For example, one of the hardest challenges in hand in finger tracking is getting reliable finger touch between the thumb and any other finger. Physical mechanical coupling and variation between hand sizes and shapes and glove manufacturing tolerances make a highly complex relationship for the machine learning model to learn when the output is the state of every degree of freedom. A model trained on just the distance between the tip of the index finger and the thumb however much more reliably predicts that separation at the expense of a decrease in accuracy for the true state of the other degrees of freedom. A specific pose or sequence can activate this model to give high fidelity control of a “pinch to zoom” capability for increased control.

In the present invention contextual sensitivity coupled with highly configurable poses with asymmetric activation and holding criteria enable us to more densely pack and even overlap state transitions and decision-making capabilities into a glove whilst at the same time decreasing occurrences of false positives or unintended outputs by plotting a path through pose space with distinct poses rather than having a hub and spoke model where every pose is 1 step from the neutral state.

Accordingly, as shown in FIG. 5 the Raw Sensor data, i.e. capacitance values may be fed into a model for predicting a hand state or gesture. In the illustrated example this is shown as a number of deployed machine learning models however this should not be seen as limiting, and as previously mentioned alternative approaches to determining hand states, poses and gestures are provided by the present technology.

Once the hand state is known, this can be matched to the closest learnt pose, subject to the pose containers and groups. For example, poses may be enabled or disabled based on context, making it easier to distinguish between similar poses. Once the pose has been selected, the visual representation in a virtual environment may be updated, control may be enacted depending on the application, and in some examples of the technology, the pose or gesture may trigger an action as described herein.

In some examples of the technology, once the pose has been recognised, this information may be fed back into the models configured to determine the hand state. For example, the reference capacitance values which correspond to a given pose may be updated during use which can help to account for factors such as the fit of the wearable device/glove. Accordingly, by limiting the number of possible poses in any given context using the pose containers and groups described herein, it may be possible to more easily detect intended poses or gestures and apply corresponding corrections to the models used to classify the detected hand positions. For example by modifying the weights of one or more models using a weight modifier.

In some examples of the technology, the recognised pose, and/or corresponding active pose containers and groups may be used to enable or disable certain models within the hand state detection system. In other words, for a given pose, such as a closed fist, models specifically trained to detect splayed fingers may be disabled, to thereby reduce the computational burden of hand position detection or classification. For battery powered wearable devices this can advantageously reduce the power draw, and extend the run time of the device.

Accordingly, in one example of the technology the processor is provided with a pre-configured set of hand positions information which are linked to one or more events. One or more of the events may include require the processor to have context information before the associated event is sent to a remote device as an event or command. For example, the following table provides an example of preconfigured hand position information which may correspond to control of a character in a virtual environment.

TABLE 1
Exemplary pre-configured hand position information

| Hand Position Information | Events | Requires |
|---|---|---|
| Clenched Fist (static) | Enter Combat | — |
| Clenched Fist (moving) | Send Punch Event | Combat |
| Relaxed hand | Holster weapon/exit combat | — |
| | mode, enter a movement mode | |
| Move from relaxed hand to | Send interact event, start | — |
| partially closed hand | interacting with object | |
| Hand in gun pose | Pick up/draw weapon, enter a | — |
| | shooting mode. | |
| Trigger finger movements in | Fire weapon | Shooting |
| gun pose | | |
| Hand moving through space | Attack/block in direction of hand | Combat |
| | movements | |
| Hand moving through space | Trigger locomotion of user | Movement |
| | according to hand movements. | |
| Hand moving through space | Move object according to hand | Interacting with |
| | movements | object |
| Hand moving through space | Aim weapon according to hand | Shooting |
| | movements. | |

In the above example a number of preconfigured sets of hand information have been provided which may correspond to a control scheme for a game. In this example, the processor is configured to determine whether the user's hand is in one of more of the pre-configured hand positions, and depending on whether the associated requirements are met, perform an action, such as sending an event to a remote device.

For example, detecting that a user has clenched their first may send an event for the game to enter a combat function. On a controller this may for example correspond to a pressing of one of the triggers such as L1 or L2. This action also provides the processor with context information such that it understands that subsequent movements of the hand should relate to attacking or blocking in the direction of the hand movements.

In some examples the processor may retain this context until another preconfigured hand position, such as moving the hand into a gun pose is detected. This can switch the context of the processor, and optionally send an event to the game to draw a gun/weapon. With this new context the processor may be configured to provide events which convert movement of the wearable device to movements or aiming of a gun.

As such, the present technology provides a wearable device such as a glove, which includes a body configured to receive the hand of a user. One or more sensors are operatively connected to the body that are configured to provide data relating to the location, orientation and/or configuration of the body in use. For example, these sensors may provide capacitance measurements corresponding to deformation of the body of the glove and/or inertial motion data corresponding to the movements and orientation of the glove. This sensor data may be processed by a processor to determine hand position information.

In order to control a remote device, the processor then compares the hand position information against a pre-configured set of hand positions which are linked to one or more events, these events can include one or more commands to be sent to a remote device together with one or more pieces of context information.

For example, the context information may be configured to switch the processor between providing events relevant to a first function (such as a locomotion) and a second function (such as interaction), whereby the events sent by the processor to a remote device with one context may differ from the events sent with a different context.

In addition, the processor may be configured to use a first algorithm or trained machine learning model to process the hand position information in a first mode or context and a second algorithm or trained machine learning model to process the hand position information in a second mode or with a second context. For example, in most situations it may be desirable for the wearable device to quickly detect and movements and send these movements as events to a remote device to allow for a responsive user experience. This can be particularly true for fast paced action games where rapid movements are desirable. However, for some control functions it may be beneficial to provide slower more precise movement, and as such the second algorithm or trained machine learning model may apply additional averaging or smoothing of the data to provide more precise control. Furthermore as described herein the movements provided with a first context may be mapped 1:1 in the virtual environment while movements provided with a second context may be mapped at a lower ratio such as 1:0.5 in order to increase the control and precision.

### 6.2.7. Control of Virtual Environments

Accordingly, using the systems method and devices described herein it may be possible to achieve more sophisticated/complex outcomes then is available using current approaches. For example, in a virtual environment, a director can be using gloves in a multimodal fashion to control the virtual environment the performers are immersed in or interacting with. A particular pose can be used to select or activate a specific function to control, e.g., the brightness of the scene lighting, the position of the lighting, the position of the virtual camera, the zoom of the virtual camera, or any number of parameters that are used in the specification of a scene. Individual degrees of freedom can then be used to adjust the activated control, e.g., a first pose could be used to activate the zoom control of the virtual camera, and then a pinching movement of the thumb and index finger can be used to control the camera zoom.

Similarly in another example, a performer may use overall proximity to specific pose to trigger a special animation such as the creation of a fireball, with subsequent or superimposed motion of individual fingers to control the size, speed and direction of the fireball when it is flung by the performer. The advantage of this approach is that the performer is controlling and, along with the crew of a virtual production can see in real time the special effects and be able to evaluate it and make adjustments in real time to their creative process.

Other applications of the present technology include:
Key frame animation—a performer or animator can wear the wearable device, and the system may be configured to only output from a pre-selected library of hand poses. The benefit of this approach is the output can only be exact desired poses, and the poses are output in sync with the performer/animator/wearer performing the hand pose.
For animation triggers—the wearer can specify an event to be triggered when a pose is in user-defined proximity to a reference pose of interest. This could be used to initiate a specific animation, e.g., a lightning bolt or a fireball.
For the capture of nuanced hand motion—by training the system with multiple reference poses, it is possible to interpolate the raw sensor data to synthesize real time predictions of intermediate hand poses that the wearer is performing at any point in time.
Virtual production—select specific controls using global poses and adjust these parameters using the movements of a subset of fingers and their relative movement.

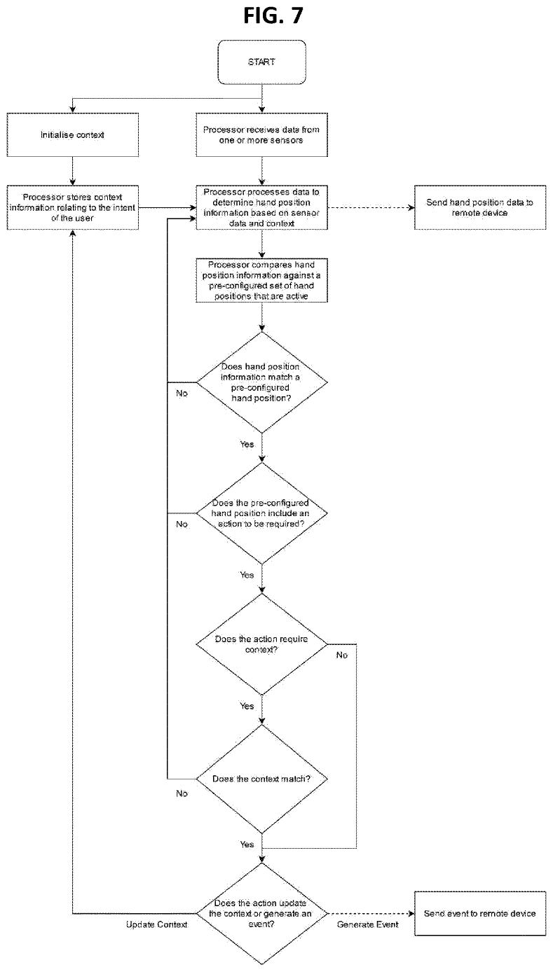

FIG. 7 shows an example of a control diagram for controlling a remote device in accordance with one example of the technology. In this example the following steps are performed:
Once the process is started, a default context may be assigned to the processor. This context may be received from a remote device, inferred by the remote device the wearable device is connected to, restored from a previous session, or may just represent a generic/default context.
The processor receives data from one or more sensors on the wearable device (such as capacitance information or IMU positional data).
The processor processes this data to determine hand position information, in some examples the processor uses this data in combination with the context information to determine how the hand position information should be processed. For example, with a first piece of context information the processor may be configured to use a first algorithm or model and with a second piece of context information, the processor may be configured to use a second algorithm or model.
In some examples this hand position information may be sent directly to a remote device. This may be done in parallel with, or separately to sending events to the remote device as described herein.
The processor compares the hand position information against a pre-configured set of hand positions to determine whether the hand position information corresponds to a pre-configured hand position.
If no match is detected, the processor continues this loop looking for hand position data that matches.
If a match is detected, the processor checks whether the pre-configured hand position corresponds to an action that needs to be performed. If so the processor checks if the action requires context information, and if so whether the context the process has matches the required context.
Finally the processor determines whether the action is to update the context information or to generate an event, and responds accordingly.
For example the event may be to sending an event to the remote device to affect an action. If at any point in the process the conditions are met, the loop can revert to processing data to determine hand position data once more.

This approach can advantageously allow for the processor to ignore pre-configured hand positions which are not relevant to the present context, while simultaneously updating the context as necessary, and sending events when the correct hand gesture or pose is performed in the correct context.

### 6.3. Exemplary Devices

### 6.3.1. Drone Control System

One advantage of the present technology is that the control systems and devices described herein may be used to control any software, electronic or electromechanical hardware. For example, one application for the technology is to provide a drone control system comprising a wearable device as described herein. For example, the user's hand position may be used to control parameters such as the drone's height, position, pitch, yaw, roll, speed, altitude and the operation of any connected devices such as cameras by using nuanced finger and hand movement, or by performing predefined gestures and poses.

### 6.3.2. Surgical Control Systems

Another application of the present technology is use in precise surgical operations. For example, the wearable device may be used to control a remote robot arm used to perform surgery on a human being or animal.

One advantage of this system is that the present technology allows, some movements of the user's hand to be ignored in use. For example, the system may be configured with a condition such as requiring that surgeon performing the operation must perform a gripping gesture (forefinger and thumb touching) when control of the remote robot is required, accordingly the remote robot may mimic the surgeon's movements with a scalpel for example. As soon as the surgeon wishes to stop controlling the robot, they can separate their forefinger and thumb, and have a high level of confidence that the robot will not move.

Furthermore, as noted herein, it is possible for the movements performed using the glove to result in control of an avatar or robot with a non 1:1 mapping of movement. For example, for every centimetre the surgeon moves, the corresponding robot may be configured to move one millimetre.

One aspect of the present technology is to provide a wearable device comprising a plurality of sensors, configured to provide position information to a processor 106. For example, the wearable device may comprise at least one capacitive stress or strain sensor for each of the joints within the user's hand, in another example the wearable device may comprise at least one capacitive stress or strain for each degree of freedom within a user's hand.

In some examples the wearable device may comprise a position tracking module 102, configured to extract the position information from the plurality of sensors 104.

### 6.3.3. Universal Input Device

In another example of the technology, the wearable device may be configured to function as universal peripheral device in a similar manner to a keyboard or mouse. Accordingly, in one example of the technology the wearable device may comprise one or more sensors configured to sense stress or strain characteristics in the wearable device, a position tracking module 102 configured to convert the stress or strain characteristics to positional and or rotational information relating to at least fingers in the user's hand, and a processor 106 configured to detect when a gesture has been performed.

For example, the wearable device may be provided with a wireless connection, such as a Bluetooth connection, which in use can connect to a device. The use of wireless connections should not be seen as limiting on the technology, and in other examples, a wired connection such as a USB connection may be used.

In some examples of the technology, when connected to a computing device such as a personal phone, tablet or computer, the wearable device may be configured to identify itself as a human interface device such as a keyboard and/or mouse. Accordingly, the present technology may be able to provide keyboard and mouse inputs to a system without requiring custom drivers or software to be installed on said system.

In use the positional and rotational information provided from the glove may be used to trigger actions on the connected computing device. For example, when controlling a presentation, a simple swiping right gesture may be used to move to the next slide, while a simple swiping left gesture could be used to transition to a previous slide. Similarly gestures familiar to those who use touch screen interfaces such as pinching to zoom may also be affected virtually in the computing device by making corresponding gestures or poses.

### 6.3.4. Sign Language Interpreting

In another example of the technology, the wearable device, may be configured to detect one or more gestures or poses which correspond to sign language, and as on detecting appropriate sign language, the wearable device may be configured to send a sequence of keypresses which correspond to the words and phrases signed using the sign language. For example the when the processor 106detects hand position information corresponding to the poses shown in FIG. 6 the processor may be configured to send the corresponding event as a keyboard key press of the corresponding letter. It should be appreciated that not all sign language systems involve gestures only, and in some examples of the technology it may be advantageous to combine the sensor information from the wearable device with camera information, such as by extracting facial gestures from the user, using any known computer vision techniques known to those skilled in the art.

For example, a condition may be added to the system whereby a sequence of hand movements does not generate a predetermined sequence of words as an output unless the corresponding facial gesture condition is also met.

### 6.3.5. Wearable Systems

One aspect of the present technology is to provide a tracking system comprising a wearable device that includes a plurality of sensors configured to provide data indicative of a stress or strain measured within the wearable device, and a position tracking module 102 configured to extract positional information from the stress or strain. In some examples the position tracking module 102 may be configured to provide the positional information to a processor to trigger an action.

### 6.4. Other Remarks

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The technology may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the technology and without diminishing its attendant advantages. It is therefore intended that such changes and modifications be included within the present technology.

### 7. DEFINITIONS

Compliant, a compliant material—a compliant material can bend, is able to stretch, and has a resilient character which returns this material to approximately its initial size and shape after each occurrence of a bending or stretching action.

Compression—as used herein the term is intended to be interpreted broadly as complying under a compressing force and is not limited to elements which change volume under pressure.

Compressible—as used herein the term is intended to be interpreted broadly as flattened by pressure.

Electronic component—as used herein the term is intended to be interpreted broadly to include any electronic component and includes circuit boards, sensors, generators, and actuators.

Electrode and terminal—as used herein are intended to be refer broadly to a point by which an electric current enters or leaves an electronic component or electrical device.

Context information—information about what the user is interacting with, for example this can be context about an application running on a remote device, or proximity to an object in a virtual environment, in other examples the context information can depend on previous actions performed by the user, such as performing a pose or gesture indicating which is pre-configured to provide the processor with different context information.

Hand position information—information regarding the positioning, rotation and orientation of the user's hand in space. This can include finger joint angles, wrist angles, angles with respect to ground, or the magnetic fields of the earth.

Remote device—any device which may be controlled or communicated with using the wearable devices described herein, for example a phone, tablet, computer, console, VR headset, home automation system, drone, robot (including for example surgical robots) etc.

Modes—this refers to distinct operating behaviours, such as whether the processor is configured to provide hand position data (for example for motion capture applications), raw capacitance data (for example for diagnostics), event data (for example when emulating a keyboard or controller), or control data (for example when manipulating robotics or home automation devices).

Events—these refer to information communicated to the remote device, for example this can include sensor data, hand position information, commands, key presses etc.

Context—this refers to an internal state or understanding the processor uses to determine how it should operate, and what information should be communicated via the remote device.

1. A wearable device comprising:
a body configured to receive a part of the anatomy of a user;
one or more sensors operatively connected to the body that are configured to provide data relating to the location, orientation and/or configuration of the body in use;
a processor configured to process the data to determine anatomical position information; and
a communications module operatively connected to the processor, and configured to communicate with at least one remote device,
wherein the processor is configured to compare the anatomical position information against a pre-configured set of anatomical positions linked to one or more events, to determine whether the anatomical position information corresponds to one of the pre-configured anatomical positions,
wherein the processor receives context information from any one or more of the remote device, the events sent to the remote device; and/or from existing context information and anatomical position data, and wherein the processor uses the context information together with the anatomical position information to determine whether the processor should send an event of the linked one or more events to the remote device via the communications module.

2. The wearable device as claimed in claim 1, wherein the processor is configured to use a first algorithm or model with a first piece of context information, and a second algorithm or model to process the data with a second piece of context information.

3. The wearable device as claimed in claim 2, wherein the first algorithm or model is an AI model trained to detect a first range of anatomical positions and wherein the second algorithm or model is an AI model trained to detect a second range of anatomical positions.

4. (canceled)

5. (canceled)

6. The wearable device as claimed in claim 1, wherein the data provided by the one or more sensors are capacitance readings relating to a configuration of the wearable device as a result of deformation in the body of the wearable device due to the user's anatomical positioning within the wearable device.

7. The wearable device as claimed in claim 1, wherein the anatomical position information is hand position information and wherein the hand position information comprises information on the relative positioning of each of the fingers of the user with respect to the palm of the hand of the user.

8. The wearable device as claimed in claim 1, wherein the anatomical position information is hand position information and wherein the hand position information includes information of the amount of bend in any one or more of the scapho-trapezium/trapezoid, the carpometacarpal, the metacarpophalangeal, the proximal Interphalangeal, and distal interphalangeal joints.

9. The wearable device as claimed in claim 1, wherein the anatomical position information includes location and/or orientation information provided by a sensor in the form of an inertial motion unit, accelerometer, gyroscope or magnetometer.

10. The wearable device as claimed in claim 1, wherein the processor is configured to send the event via the communications module to the remote device.

11. The wearable device as claimed in claim 1 wherein, the processor is configured to determine whether the anatomical position information corresponds to a pre-configured anatomical position using a machine learning model;
wherein the machine learning model is configured to determine whether the anatomical position information corresponds to a pre-configured anatomical position using a neural network;
wherein the neural network provides a confidence score indicating the likely match to any one of the pre-configured anatomical positions;
wherein the processor is configured to determine whether the anatomical position information corresponds to one of the pre-configured set of anatomical positions when the confidence score exceeds a predetermined threshold;
wherein the processor is configured to send the event or update the context information when the confidence score for one of the pre-configured anatomical positions is higher than the confidence score for any other of the pre-configured anatomical positions.

12-15. (canceled)

16. The wearable device as claimed in claim 1, wherein the processor is further configured to switch between a first mode in which anatomical position data is communicated with the remote device, and a second mode, wherein the events are communicated with the remote device.

17. The wearable device as claimed in claim 16, wherein the first mode and second mode are active simultaneously.

18. The wearable device as claimed in claim 16, wherein the processor is configured to switch between, enable or disable either of the first mode and the second mode when a sequence of anatomical positions are detected which correspond to a pre-configured sequence of anatomical positions associated with a change of operating mode.

19. The wearable device as claimed in claim 16, wherein the processor is configured to send the event or update the context information if the pre-configured anatomical position is detected for a predetermined period of time.

20. The wearable device as claimed in claim 19, wherein the event comprises one or more of: a keypress; a multi-media command; an animation event; an augmented/virtual/mixed reality event; displacement on one more axes; anatomic position and/or orientation data; a biometric identification event; an authorization event; or a user-defined event.

21. The wearable device as claimed in claim 19, wherein the event comprises orientation information from an inertial motion unit, accelerometer, gyroscope or magnetometer.

22. The wearable device as claimed in claim 1:
wherein the processor is configured to operate in:
a first mode in which the anatomical position information is provided to the communications module for communication with the remote device; and
a second mode in which the processor compares the anatomical position information against a pre-configured set of anatomical positions, wherein when the anatomical position information is determined to correspond to a pre-configured anatomical position, the processor is configured to send an event to the communications module for communicating the event to the remote device or to update the context information.

23-39. (canceled)

40. A processor-implemented method of controlling a remote device using at least one wearable device which comprises, a body configured to receive a part of the anatomy of a user, one or more sensors operatively connected to the body that are configured to provide data relating to the location, orientation and/or configuration of the body in use, a processor configured to process the data to determine anatomical position information, and a communications module operatively connected to the processor, the method comprising the steps of:
in a first mode of operation:
A) processing the data from the one or more sensors to provide anatomical position information;
B) comparing the anatomical position information to a pre-configured set of anatomical positions linked to one or more events;
C) determining when the anatomical position information corresponds to a pre-configured anatomical position;
D) communicating the corresponding events to the remote device using the communications module to thereby control the remote device;
and in a second mode of operation:
E) processing the data from the one or more sensors to provide anatomical position information; and
F) communicating the anatomical position information to the remote device using the communications module to thereby control the remote device.

41. The processor-implemented method of claim 40, wherein one or more of the pre-configured set of anatomical positions provides the processor with context information about the intended behaviour of the user.

42. The processor-implemented method of claim 41, wherein with a first context the processor is configured to use a first algorithm or model to process the data and with a second context, the processor is configured to use a second algorithm or model to process the data.

43. A wearable device comprising:
a body configured to receive a part of the anatomy of a user;
one or more sensors operatively connected to the body that are configured to provide data relating to the location, orientation and/or configuration of the body in use;
a processor configured to process the data to determine whether anatomical position information corresponds to at least one of a set of predetermined anatomical positions each of the predetermined anatomical positions being linked to one or more events; and
a communications module operatively connected to the processor, and configured to communicate with at least one remote device,
wherein the processor is configured to receive context information from the remote device, and based on the context information enable or disable one or more of the events, such that after receiving a first piece of context information, the processor is configured to select from a first list of events corresponding to the predetermined anatomical positions, and after receiving a second piece of context information the processor is configured to select from a second list of events corresponding to the predetermined anatomical positions.

44-46. (canceled)
